(19) United States
(12) Patent Application Publication
(10) Pub. No.: US 2013/0018155 A1
(43) Pub. Date: Jan. 17, 2013

(54) METAL COMPOSITES AND COMPOUND USEFUL FOR PREPARATION THEREOF

(71) Applicants: Kenta Tanaka, Tsukuba-shi (JP); Masahiro Fujioka, Wakayama-shi (JP)

(72) Inventors: Kenta Tanaka, Tsukuba-shi (JP); Masahiro Fujioka, Wakayama-shi (JP)

(73) Assignee: SUMITOMO CHEMICAL COMPANY, LIMITED, Chuo-ku, Tokyo (JP)

(21) Appl. No.: 13/583,154

(22) PCT Filed: Mar. 31, 2011
(86) PCT No.: PCT/JP2011/058746

§ 371 Date: Sep. 24, 2012

(30) Foreign Application Priority Data
Apr. 5, 2010 (JP) 2010-086738

(51) Int. Cl. C07D 251/18; C08G 75/00; C08G 61/02

(52) U.S. Cl. 525/535; 544/219; 528/373; 528/380

(57) ABSTRACT

A polymer compound having a constituent unit represented by the following formula (P-a) and having a molecular weight of 5×10² to 1×10⁷:

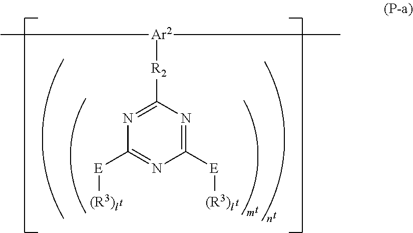

(wherein Ar² represents an aromatic group optionally having a substituent, R² represents a direct bond or an organic group optionally having only an oxygen atom as a hetero atom, E represents a hetero atom, R³ represents a monovalent hydrocarbon group or a hydrogen atom, m^{t} and n^{t} represent each independently an integer of 1 or more, and l^{t} represents an integer of 1 to 3. Each of a plurality of R³s, Es and l^{t}s may be mutually the same or different. When there exist a plurality of m^{t}s, these may be mutually the same or different. When there exist a plurality of groups in parentheses appended with m^{t} and n^{t}, these may be mutually the same or different.).

### TECHNICAL FIELD

The present invention relates to a metal composite and a compound which is useful for preparation thereof.

### BACKGROUND ART

A conjugated compound having properties such as thermal conductivity, electric conductivity, heat resistance and the like is combined with a metal to make up for each other's weak points and to manifest a novel function, thus, a metal composite obtained by such combination attracts attention as a material of the next generation. For producing the metal composite, it is indispensable that a conjugated compound and a metal, which are different materials, are allowed to adsorb strongly with good efficiency (Non-patent document 1: J. E. Katon, Polymer Organic Semiconductor (kobunshi yuki handoutai), chapter 2).

### SUMMARY OF THE INVENTION

Thus, the present invention has an object of providing a compound which is capable of adsorbing strongly to a metal with good efficiency, and a metal composite using the same.

The present invention provides, in a first aspect, a compound represented by the following formula (II-a):

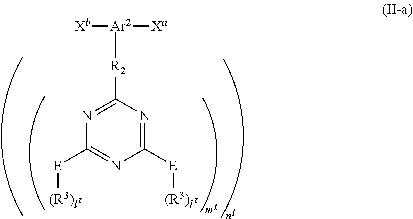

(wherein Ar² represents an aromatic group optionally having a substituent, R² represents a direct bond or an organic group optionally having only an oxygen atom as a hetero atom, E represents a hetero atom, R³ represents a monovalent hydrocarbon group or a hydrogen atom, m^{t} and n^{t} represent each independently an integer of 1 or more, l^{t} represents an integer of 1 to 3. Each of a plurality of R³s, Es and l^{t}s may be mutually the same or different. When there exist a plurality of m^{t}s, these may be mutually the same or different. X^{a} and X^{b} represent each independently a halogen atom, a nitro group, —SO₃Q (here, Q represents an unsubstituted or substituted monovalent hydrocarbon group.), —B(OQ¹)₂ (here, Q¹ represents a hydrogen atom or a monovalent hydrocarbon group, alternatively two Q¹s are linked together to form a ring. Two Q¹s may be mutually the same or different.), —B(OQ^{0 1})₃.M^{a} (wherein, Q^{0 1} represents a hydrogen atom or a monovalent hydrocarbon group, alternatively two to three Q⁰¹s are linked together to form a ring. Three Q⁰¹s may be mutually the same or different. M^{a} represents a metal cation or an ammonium cation optionally having a substituent.), —Si(Q²)₃ (here, Q² represents a monovalent hydrocarbon group.) or —Sn(Q³)₃ (here, Q³ represents a monovalent hydrocarbon group.). When there exist a plurality of groups in parentheses appended with m^{t} and n^{t}, these may be mutually the same or different.).

The present invention provides, in a second aspect, a polymer compound having a constituent unit represented by the following formula (P-a) and having a molecular weight of 5×10² to 1×10⁷:

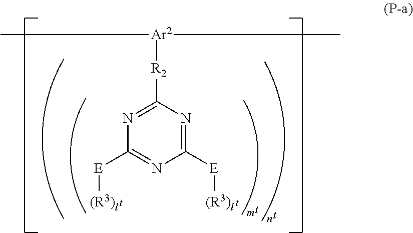

(wherein Ar² represents an aromatic group optionally having a substituent, R² represents a direct bond or an organic group optionally having only an oxygen atom as a hetero atom, E represents a hetero atom, R³ represents a monovalent hydrocarbon group or a hydrogen atom, m^{t} and n^{t} represent each independently an integer of 1 or more, and l^{t} represents an integer of 1 to 3. Each of a plurality of R³s, Es and l^{t}s may be mutually the same or different. When there exist a plurality of m^{t}s, these may be mutually the same or different. When there exist a plurality of groups in parentheses appended with m^{t} and n^{t}, these may be mutually the same or different.).

The present invention provides, in a third aspect, a metal composite obtained by bringing the above-described polymer compound into contact with a metal in the form of film or plate or with a metal compound in the form of film or plate.

The present invention provides, in a fourth aspect, a metal composite obtained by bringing the above-described polymer compound into contact with metal nano particles having an aspect ratio of less than 1.5 or with metal compound nano particles having an aspect ratio of less than 1.5.

The present invention provides, in a fifth aspect, an electron device comprising the above-described metal composite.

According to the present invention, a compound which is capable of adsorbing strongly to a metal with good efficiency, and a metal composite using the same are obtained. The compound of the present invention is particularly useful as a raw material of advanced functional materials such as electron composite materials and the like.

### MODES FOR CARRYING OUT THE INVENTION

Next, the present invention will be explained in detail.

In the present specification, “adsorption” means chemical adsorption and physical adsorption.

### <Compound>

The compound of the present invention is a compound represented by the above-described formula (II-a).

The aromatic group represented by Ar² in the above-described formula (II-a) includes atomic groups remaining after removal of two hydrogen atoms from compounds represented by the following formulae (1) to (91), and the like. This aromatic group may have a substituent.

Among compounds represented by the following formulae (1) to (91), compounds represented by the formulae (1) to (12), (15) to (22), (24) to (31), (37) to (40), (43) to (46), (49), (50), (59) to (76) are preferable, compounds represented by the formulae (1) to (3), (8) to (10), (15) to (21), (24) to (31), (37), (39), (43) to (45), (49), (50), (59) to (76) are more preferable, compounds represented by the formulae (1) to (3), (8), (10), (15), (17), (21), (24), (30), (59), (60) and (61) are further preferable, compounds represented by the formulae (1) to (3), (8), (10) and (59) are particularly preferable, since synthesis thereof is easy.

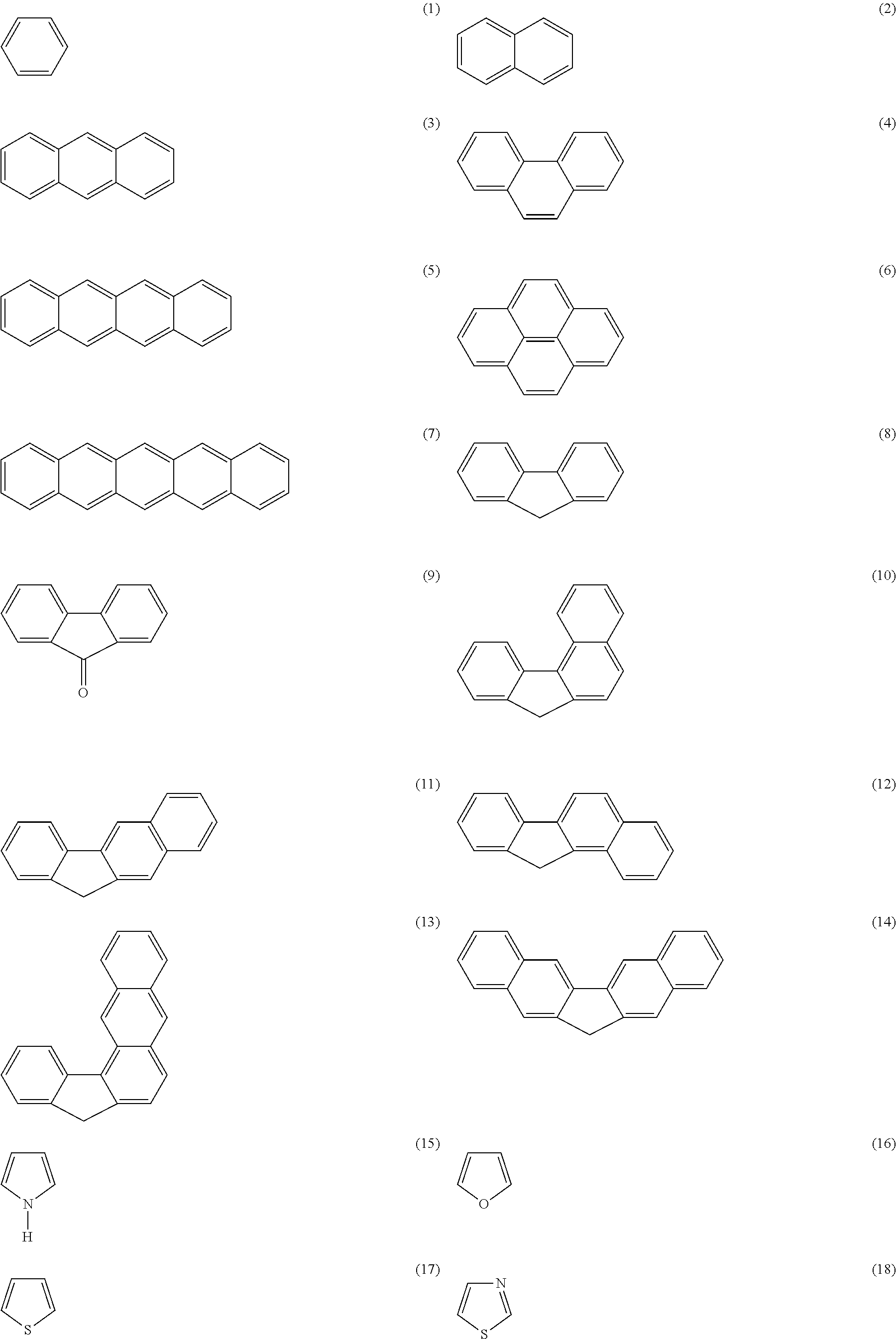

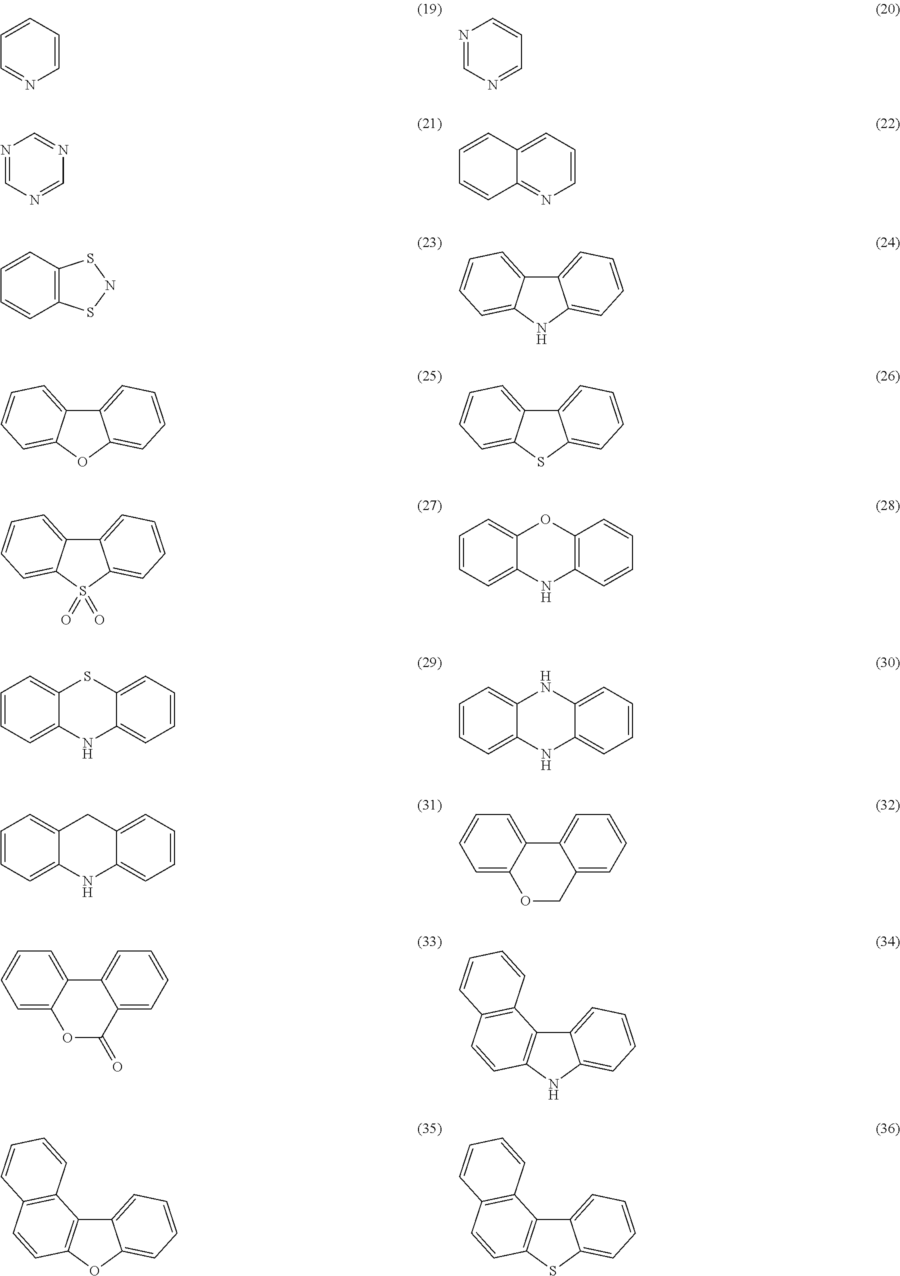

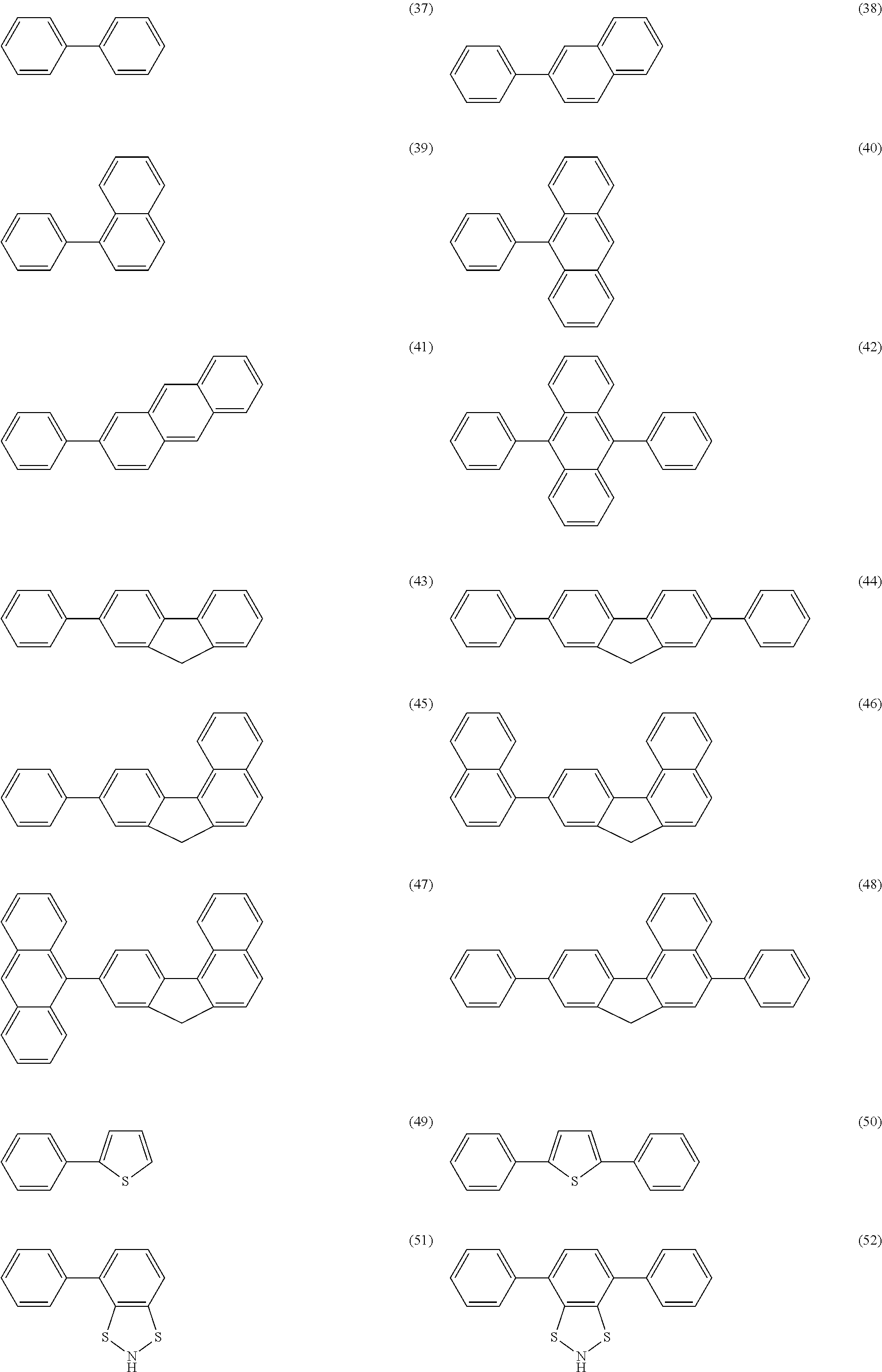

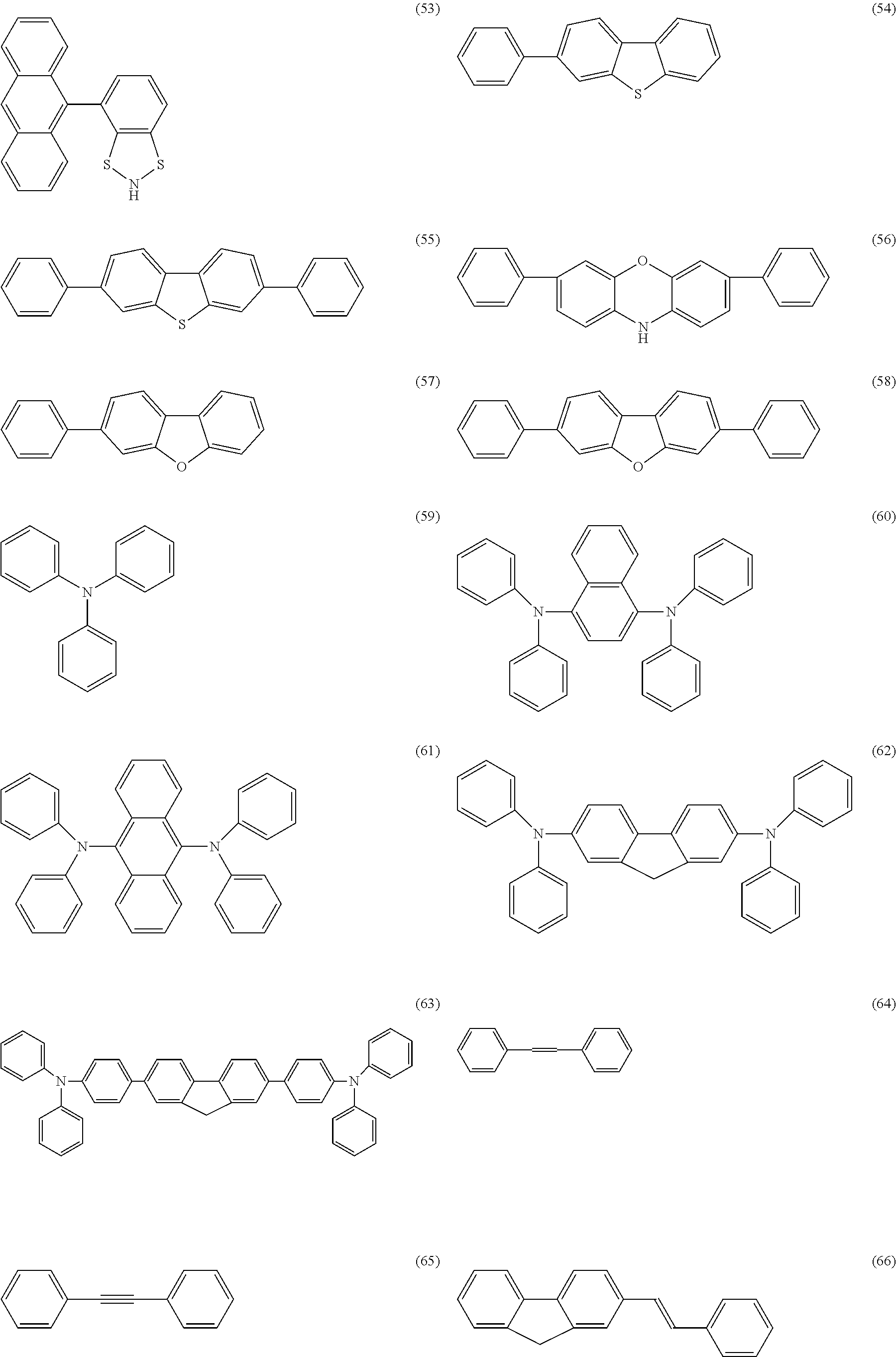

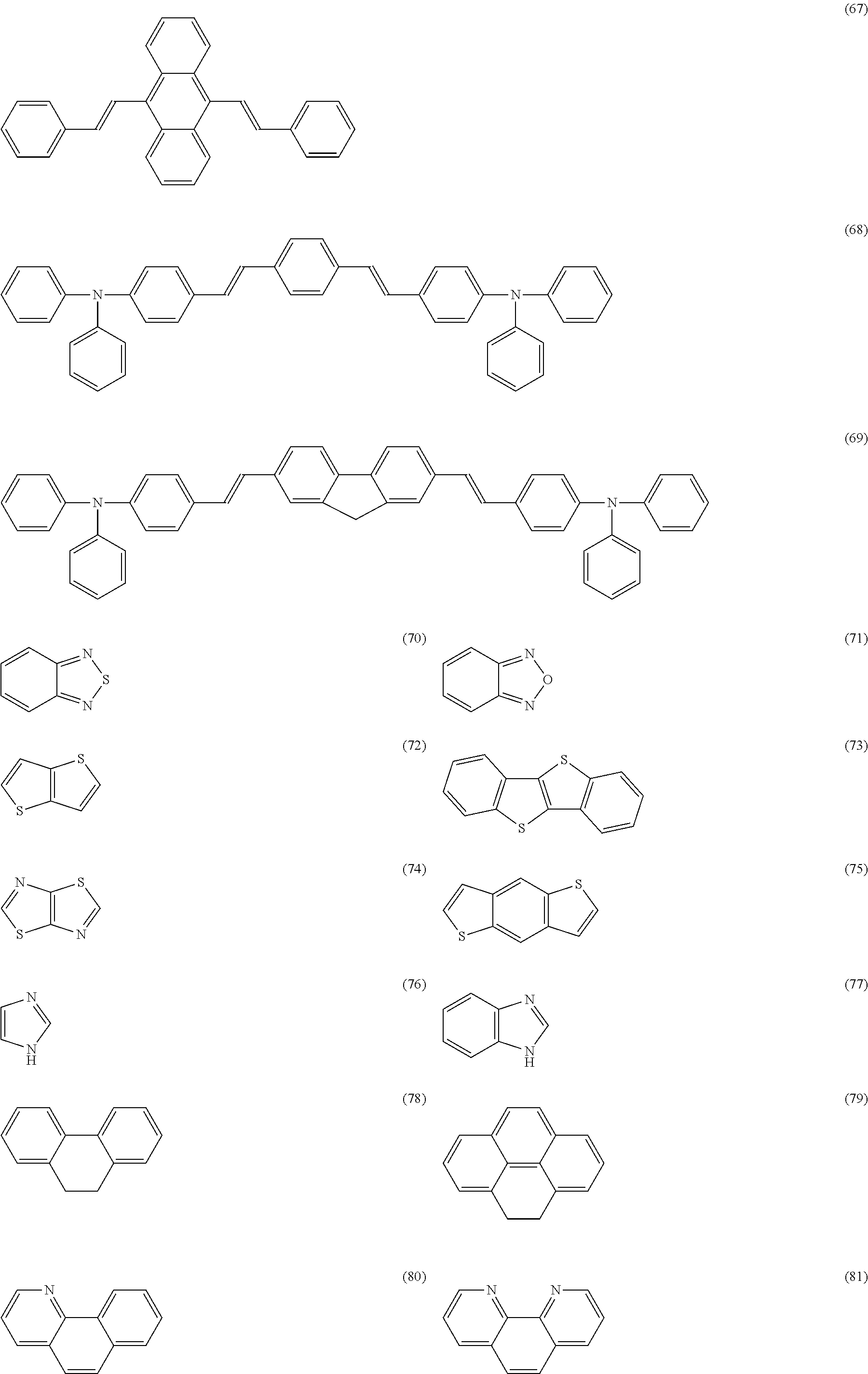

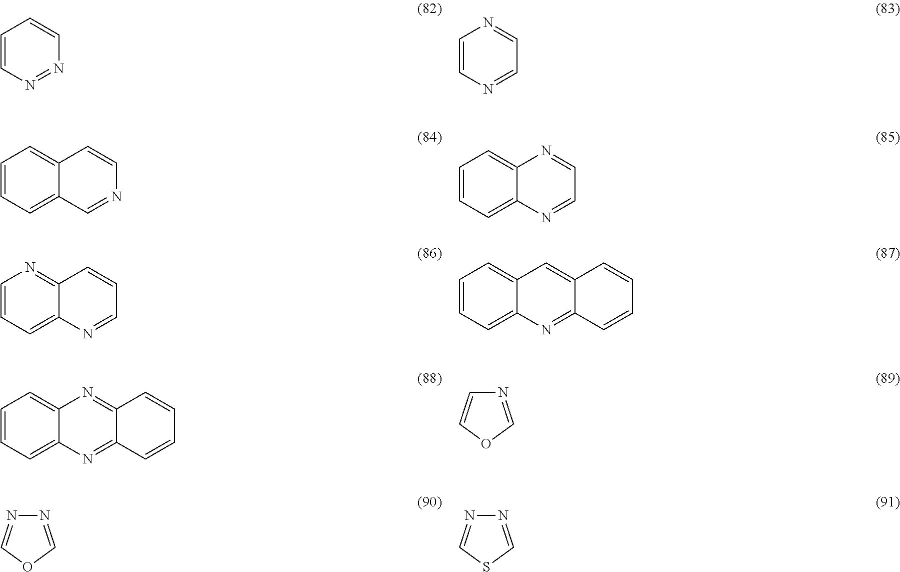

The substituent optionally carried on the above-described aromatic group represented by Ar² includes a halogen atom, a monovalent hydrocarbon group optionally having a substituent, a mercapto group, a carbonylmercapto group, a thiocarbonylmercapto group, a hydrocarbon thio group optionally having a substituent, a hydrocarbon thiocarbonyl group optionally having a substituent, a hydrocarbon dithio group optionally having a substituent, a hydroxyl group, a hydrocarbonoxy group optionally having a substituent, a carboxyl group, an aldehyde group, a hydrocarboncarbonyl group optionally having a substituent, a hydrocarbonoxycarbonyl group optionally having a substituent, a hydrocarboncarbonyloxy group optionally having a substituent, a cyano group, a nitro group, an amino group, a hydrocarbon mono-substituted amino group optionally having a substituent, a hydrocarbon di-substituted amino group optionally having a substituent, a phosphino group, a hydrocarbon mono-substituted phosphino group optionally having a substituent, a hydrocarbon di-substituted phosphino group optionally having a substituent, a formula: —P(═O)(OH)₂, a carbamoyl group, a hydrocarbon mono-substituted carbamoyl group optionally having a substituent, a hydrocarbon di-substituted carbamoyl group optionally having a substituent, a group represented by the formula: —B(OH)₂, a borate residue, a sulfo group, a hydrocarbonsulfo group optionally having a substituent, a hydrocarbonsulfonyl group optionally having a substituent, a monovalent heterocyclic group optionally having a substituent, a hydrocarbon group having two or more ether bonds, a hydrocarbon group having two or more ester bonds, a hydrocarbon group having two or more amide bonds, a group represented by the formula: —CO₂M, a group represented by the formula: —PO₃M, a group represented by the formula: —PO₂M, a group represented by the formula: —PO₃M₂, a group represented by the formula: —OM, a group represented by the formula: —SM, a group represented by the formula: —B(OM)₂, a group represented by the formula: —SO₃M, a group represented by the formula: —SO₂M (wherein M represents a metal cation or an ammonium cation optionally having a substituent.), a group represented by the formula: —NR₃M′, a group represented by the formula: —BR₃M′, a group represented by the formula: —PR₃M′, a group represented by the formula: —SR₂M′ (wherein R represents a monovalent hydrocarbon group and M′ represents an anion.), and a monovalent heterocyclic group having a quaternized nitrogen atom in the heterocyclic ring and optionally having a substituent, and the like, preferably a halogen atom, a hydrocarbon group optionally having a substituent, a mercapto group, a hydrocarbon thio group optionally having a substituent, a hydrocarbon dithio group optionally having a substituent, a hydroxyl group, a hydrocarbonoxy group optionally having a substituent, a carboxyl group, a hydrocarboncarbonyl group optionally having a substituent, a cyano group, an amino group, a hydrocarbon mono-substituted amino group optionally having a substituent, a hydrocarbon di-substituted amino group optionally having a substituent, a formula: —P(═O)(OH)₂, a sulfo group, a monovalent heterocyclic group optionally having a substituent, a group represented by the formula: —CO₂M, a group represented by the formula: —PO₃M, a group represented by the formula: —SO₃M or a group represented by the formula: —NR₃M′, more preferably a halogen atom, a hydrocarbon group optionally having a substituent, a mercapto group, a hydroxyl group, a carboxyl group, a cyano group, an amino group, a formula: —P(═O)(OH)₂, a sulfo group, a monovalent heterocyclic group optionally having a substituent, a group represented by the formula: —CO₂M, a group represented by the formula: —PO₃M and a group represented by the formula: —NR₃M′, particularly preferably a hydrocarbon group optionally having a substituent, a mercapto group, a carboxyl group, a monovalent heterocyclic group optionally having a substituent and a group represented by the formula: —CO₂M.

The above-described aromatic group represented by Ar² may have only one of or two or more of these substituents. When there are a plurality of substituents, these may together form a ring.

The substituent halogen atom includes a fluorine atom, a chlorine atom, a bromine atom and an iodine atom, preferably a fluorine atom, a chlorine atom and a bromine atom, further preferably a chlorine atom and a bromine atom.

The substituent monovalent hydrocarbon group optionally having a substituent includes alkyl groups having 1 to 50 carbon atoms such as a methyl group, an ethyl group, a propyl group, an isopropyl group, a butyl group, an isobutyl group, a t-butyl group, a pentyl group, a hexyl group, a nonyl group, a dodecyl group, a pentadecyl group, an octadecyl group, a docosyl group and the like; cyclic saturated hydrocarbon groups having 3 to 50 carbon atoms such as a cyclopropyl group, a cyclobutyl group, a cyclobutyl group, a cyclopentyl group, a cyclohexyl group, a cyclononyl group, a cyclododecyl group, a norbornyl group, an adamantly group and the like; alkenyl groups having 2 to 50 carbon atoms such as an ethenyl group, a propenyl group, a 3-butenyl group, a 2-butenyl group, a 2-pentenyl group, a 2-hexenyl group, a 2-nonenyl group, a 2-dodecenyl group and the like; aryl groups having 6 to 50 carbon atoms such as a phenyl group, a 1-naphthyl group, a 2-naphthyl group, a 2-methylphenyl group, a 3-methylphenyl group, a 4-methylphenyl group, a 4-ethylphenyl group, a 4-propylphenyl group, a 4-isopropylphenyl group, a 4-butylphenyl group, a 4-t-butylphenyl group, a 4-hexylphenyl group, a 4-cyclohexylphenyl group, a 4-adamantylphenyl group, a 4-phenylphenyl group and the like; and aralkyl groups having 7 to 50 carbon atoms such as a phenylmethyl group, a 1-phenyleneethyl group, a 2-phenylethyl group, a 1-phenyl-1-propyl group, a 1-phenyl-2-propyl group, a 2-phenyl-2-propyl group, a 3-phenyl-1-propyl group, a 4-phenyl-1-butyl group, a 5-phenyl-1-pentyl group, a 6-phenyl-1-hexyl group and the like, preferably alkyl groups having 1 to 50 carbon atoms and aryl groups having 6 to 50 carbon atoms, more preferably alkyl groups having 1 to 12 carbon atoms and aryl groups having 6 to 18 carbon atoms, particularly preferably alkyl groups having 1 to 6 carbon atoms and aryl groups having 6 to 12 carbon atoms.

The hydrocarbon thio group optionally having a substituent, the hydrocarbon thiocarbonyl group optionally having a substituent, the hydrocarbon dithio group optionally having a substituent, the hydrocarbonoxy group optionally having a substituent, the hydrocarboncarbonyl group optionally having a substituent, the hydrocarbonoxycarbonyl group optionally having a substituent and the hydrocarboncarbonyloxy group optionally having a substituent, which are substituents, are groups in which a part or all (particularly 1 to 3, especially 1 or 2) of hydrogen atoms constituting each group may be substituted by the above-described “monovalent hydrocarbon group optionally having a substituent”.

The hydrocarbon mono-substituted amino group, the hydrocarbon di-substituted amino group, the hydrocarbon mono-substituted phosphino group, the hydrocarbon di-substituted phosphino group, the hydrocarbon mono-substituted carbamoyl group and the hydrocarbon di-substituted carbamoyl group, which are substituents, are groups in which one or two of hydrogen atoms constituting each group may be substituted by the above-described “monovalent hydrocarbon group optionally having a substituent”.

The substituent borate residue includes, for example, groups represented by the following formulae.

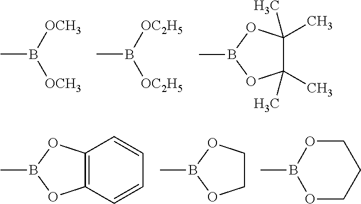

The substituent monovalent heterocyclic group is an atomic group remaining after removal of one hydrogen atom from a heterocyclic compound. The heterocyclic compound includes monocyclic heterocyclic compounds such as pyridine, 1,2-diazine, 1,3-diazine, 1,4-diazine, 1,3,5-triazine, furan, pyrrole, thiophene, pyrazole, imidazole, oxazole, thiazole, oxadiazole, thiadiazole, azadiazole and the like; condensed polycyclic heterocyclic compounds obtained by condensation of two or more heterocyclic rings constituting monocyclic heterocyclic compounds; bridged polycyclic heterocyclic compounds having a structure bridging two heterocyclic rings constituting a monocyclic heterocyclic compound or bridging one aromatic ring with one heterocyclic ring constituting a monocyclic heterocyclic compound via a divalent group such as a methylene group, an ethylene group, a carbonyl group and the like; etc., preferably pyridine, 1,2-diazine, 1,3-diazine, 1,4-diazine and 1,3,5-triazine, more preferably pyridine and 1,3,5-triazine.

The substituent hydrocarbon group having two or more ether bonds includes, for example, groups represented by the following formulae.

—R′—(OR′)ₙ—H —R′—(OR′)ₙ—OH —O—(R′O)ₙ—H —O—(R′O)ₙ—R′H

(wherein R′ represents a divalent hydrocarbon group optionally having a substituent. n is an integer of 2 or more. A plurality of R's may be mutually the same or different.).

The divalent hydrocarbon group represented by R′ includes divalent saturated hydrocarbon groups having 1 to 50 carbon atoms such as a methylene group, an ethylene group, a 1,2-propylene group, a 1,3-propylene group, a 1,2-butylene group, a 1,3-butylene group, a 1,4-butylene group, a 1,5-pentylene group, a 1,6-hexylene group, a 1,9-nonylene group, a 1,12-dodecylene group and the like; divalent unsaturated hydrocarbon groups having 2 to 50 carbon atoms such as alkenylene groups such as an ethenylene group, a propenylene group, a 3-butenylene group, a 2-butenylene group, a 2-pentenylene group, a 2-hexenylene group, a 2-nonenylene group, a 2-dodecenylene group and the like, and an ethynylene group and the like; divalent cyclic saturated hydrocarbon groups having 3 to 50 carbon atoms such as a cyclopropylene group, a cyclobutylene group, a cyclopentylene group, a cyclohexylene group, a cyclononylene group, a cyclododecylene group, a norbonylene group, an adamantylene group and the like; arylene groups having 6 to 50 carbon atoms such as a 1,3-phenylene group, a 1,4-phenylene group, a 1,4-naphthylene group, a 1,5-naphthylene group, a 2,6-naphthylene group, a biphenyl-4,4′-diyl group and the like; etc. A hydrogen atom in these groups may be substituted by a substituent.

The substituent hydrocarbon group having two or more ester bonds includes, for example, groups represented by the following formulae.

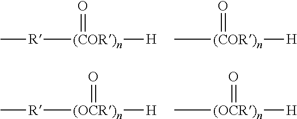

(wherein R′ and n have the same meaning as described above.).

The substituent hydrocarbon group having two or more amide bonds includes, for example, groups represented by the following formulae.

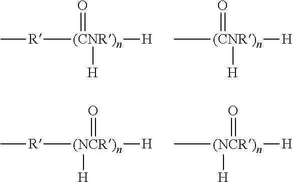

(wherein R′ and n have the same meaning as described above.).

The above-described metal cation represented by M includes preferably 1 to 3-valent ions, and includes ions of metals such as Li, Na, K, Cs, Be, Mg, Ca, Ba, Ag, Al, Bi, Cu, Fe, Ga, Mn, Pb, Sn, Ti, V, W, Y, Yb, Zn, Zr and the like.

In the above-described ammonium cation represented by M optionally having a substituent, the substituent includes alkyl groups having 1 to 10 carbon atoms such as a methyl group, an ethyl group, a propyl group, an isopropyl group, a butyl group, an isobutyl group, a t-butyl group and the like.

The above-described monovalent hydrocarbon group represented by M includes an alkyl group, a cycloalkyl group, an aryl group, an aralkyl group and the like.

The above-described anion represented by M′ includes F⁻, Cl⁻, Br⁻, I⁻, OH⁻, ClO⁻, ClO₂⁻, ClO₃⁻, ClO₄⁻, SCN⁻, CN⁻, NO₃⁻, SO₄²⁻, HSO₄⁻, PO₄³⁻, HPO₄²⁻, H₂PO₄₋, BF₄₋, PF₆⁻, CH₃SO₃⁻, CF₃SO₃⁻, a tetrakis(imidazolyl)borate anion, a 8-quinolinolato anion, a 2-methyl-8-quinolinolato anion, a 2-phenyl-8-quinolinolato anion and the like.

The substituent monovalent heterocyclic group having a quaternized nitrogen atom in its heterocyclic ring includes groups represented by the following formulae.

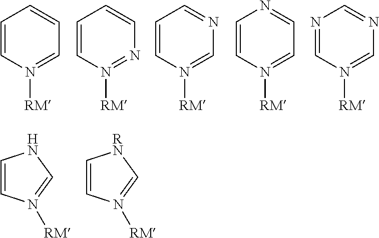

(wherein R and M′ have the same meaning as described above.).

The organic group represented by R² optionally having only an oxygen atom as a hetero atom in the above-described formula (II-a) includes an atomic group remaining after removal of a part of hydrogen atoms from a group obtained by substitution of CH₂ of the above-described monovalent hydrocarbon group optionally having a substituent by an oxygen atom, and an atomic group remaining after removal of a part of hydrogen atoms from the above-described monovalent hydrocarbon group optionally having a substituent, and these groups may mutually form a ring. R² includes preferably an atomic group remaining after removal of a part of hydrogen atoms from an alkyl group optionally having a substituent and an atomic group remaining after removal of a part of hydrogen atoms from an aryl group optionally having a substituent, more preferably an atomic group remaining after removal of a part of hydrogen atoms from an alkyl. group having 1 to 12 carbon atoms and an atomic group remaining after removal of a part of hydrogen atoms from a phenyl group, further preferably an atomic group remaining after removal of a part of hydrogen atoms from an alkyl group having 1 to 6 carbon atoms and an atomic group remaining after removal of a part of hydrogen atoms from a phenyl group.

The hetero atom represented by E in the above-described formula (II-a) includes an oxygen atom, a sulfur atom, a nitrogen atom, a phosphorus atom, a silicon atom, a selenium atom and a tellurium atom, preferably an oxygen atom, a sulfur atom and a nitrogen atom, further preferably an oxygen atom and a sulfur atom, particularly preferably a sulfur atom.

When E is an oxygen atom or a sulfur atom and R³ is a hydrogen atom, an isomerization reaction represented by the following reaction formula may occur. Also a compound generated in this isomerization reaction exerts the same effect as that of the inventive compound.

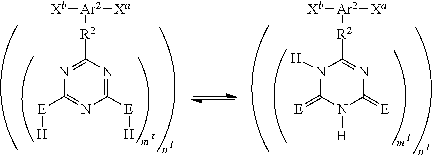

When E is a sulfur atom and R³ is a hydrogen atom, -E-H moieties of two molecules tend to mutually react to generate a structure of -E-E-. Also a compound generated in this reaction exerts the same effect as that of the inventive compound.

In the above-described formula (II-a), the monovalent hydrocarbon group represented by R³ is the same as the monovalent hydrocarbon group optionally having a substituent explained and exemplified in the above-described column of the substituent. A plurality of R³s may be mutually the same or different, and a plurality of R³s may mutually form a ring.

In the above-described formula (II-a), the halogen atom represented by X^{a} and X^{b} includes a fluorine atom, a chlorine atom, a bromine atom and an iodine atom, preferably a chlorine atom, a bromine atom and an iodine atom.

In —SO₃Q represented by X^{a} and X^{b}, the substituted or unsubstituted monovalent hydrocarbon group represented by Q is the same as the monovalent hydrocarbon group optionally having a substituent explained and exemplified in the above-described column of the substituent. Here, the substituent includes a fluorine atom.

—SO₃Q represented by X^{a} and X^{b} includes a methanesulfonate group, a benzenesulfonate group, a p-toluenesulfonate group and a trifluoromethanesulfonate group.

In —B(OQ¹)₂ and B(OQ_{0 1})₃.M^{a} represented by X^{a} and X^{b}, the monovalent hydrocarbon group represented by Q¹ or Q^{0 1} includes monovalent hydrocarbon groups optionally having a substituent explained and exemplified in the above-described column of the substituent, preferably alkyl groups, more preferably a methyl group, an ethyl group, a propyl group, an isopropyl group, a butyl group, an isobutyl group, a pentyl group, a hexyl group and a nonyl group, further preferably a methyl group, an ethyl group, a propyl group, a butyl group, a pentyl group and a hexyl group. When two Q¹s together form a ring, the divalent hydrocarbon group composed of two Q¹s includes preferably a 1,2-ethylene group, a 1,1,2,2-tetramethyl-1,2-ethylene group, a 1,3-propylene group, a 2,2-dimethyl-1,3-propylene group and a 1,2-phenylene group.

In —B(OQ^{0 1})³.M^{a} represented by X^{a} and X^{b}, M^{a} is the same as the above-described moiety explained and exemplified as M.

In —Si(Q²)₃ and —Sn(Q³)₃ represented by X^{a} and X^{b}, the monovalent hydrocarbon group represented by Q² and Q³ includes monovalent hydrocarbon groups optionally having a substituent explained and exemplified in the above-described column of the substituent, preferably alkyl groups, more preferably a methyl group, an ethyl group, a propyl group, an isopropyl group, a butyl group, an isobutyl group, a pentyl group, a hexyl group and a nonyl group, further. preferably a methyl group, an ethyl group, a propyl group, a butyl group, a pentyl group and a hexyl group.

In the above-described formula (II-a), X^{a} and X^{b} include preferably a halogen atom, —SO₃Q, —B(OQ¹)₂ and —B(OQ^{0 1})₃.M^{a}, more preferably a chlorine atom, a bromine atom, an iodine atom and —SO₃Q, further preferably a chlorine atom, a bromine atom, an iodine atom and a trifluoromethanesulfonate group, particularly preferably a chlorine atom, a bromine atom and an iodine atom, especially preferably a bromine atom.

In the above-described formula (II-a), l^{t} is an integer of 1 to 3. When E is a silicon atom, l^{t} representing the number of R³ directly linking to E is 3, when E is a nitrogen atom or a phosphorus atom, l^{t} representing the number of R³ directly linking to E is 2, and when E is an oxygen atom, a sulfur atom, a selenium atom or a tellurium atom, l^{t} representing the number of R³ directly linking to E is 1.

Among compounds represented by the above-described formula (II-a), preferable are compounds represented by the following formula (II-b), more preferable are compounds represented by the following formula (II-c).

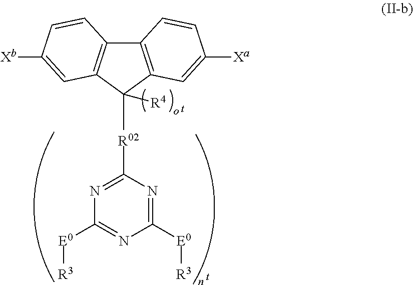

(wherein R^{0 2} represents a divalent hydrocarbon group, and E⁰ represents a sulfur atom or an oxygen atom. R⁴ represents a monovalent hydrocarbon group or a hydrogen atom, n^{t} is 1 or 2, and o^{t}=2−n^{t}. Each of a plurality of R³s and E⁰s may be mutually the same or different. When there are a plurality of R^{0 2}s, these may be mutually the same or different. R³, X^{a} and X^{b} have the same meaning as described above. When there exist a plurality of groups in parentheses appended with n^{t}, these may be mutually the same or different.).

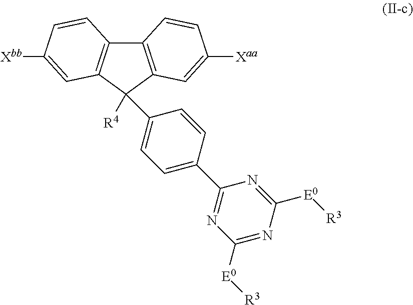

(wherein R³, R⁴ and E⁰ have the same meaning as described above. X^{a a} and X^{b b} represent each independently a chlorine atom, a bromine atom or an iodine atom.).

The divalent hydrocarbon group represented by R^{0 2} in the above-described formula (II-b) is the same as the divalent hydrocarbon group explained and exemplified in the above-described column of the divalent hydrocarbon group represented by R′, and a phenylene group is preferable.

The halogen atom represented by X^{a} and X^{b} in the above-described formula (II-b) includes a fluorine atom, a chlorine atom, a bromine atom and an iodine atom, preferably a chlorine atom, a bromine atom and an iodine atom.

The monovalent hydrocarbon group represented by R⁴ in the above-described formulae (II-b) and (II-c) is the same as the monovalent hydrocarbon group optionally having a substituent explained and exemplified in the above-described column of the substituent.

Examples of the compound of the present invention include compounds represented by the following formulae (F-1) to (F-29), preferably compounds represented by the following formulae (F-1) to (F-12).

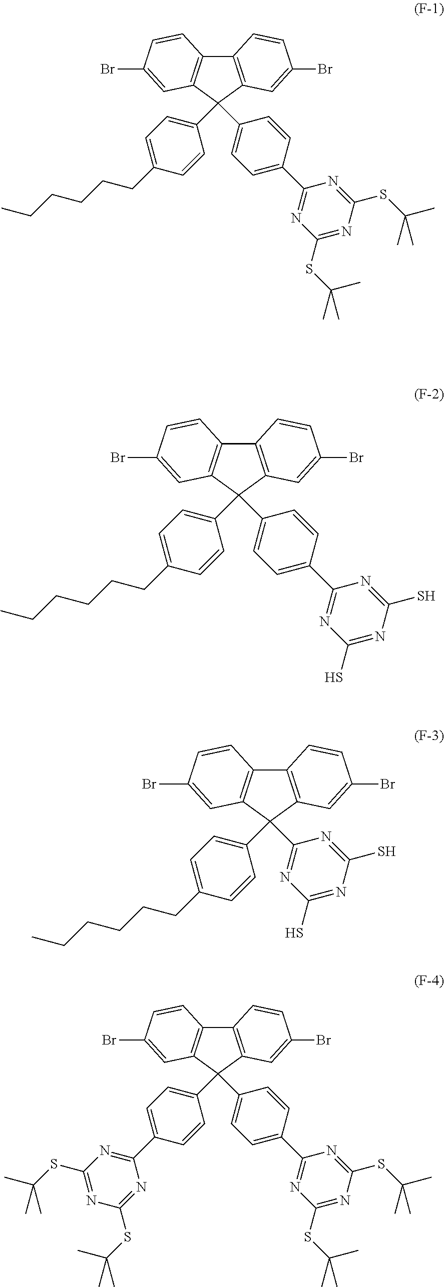

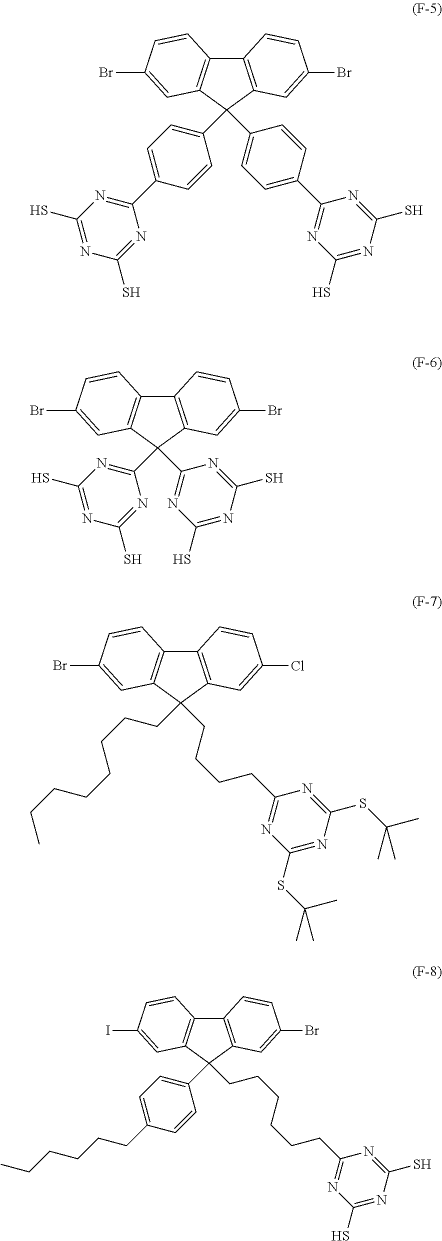

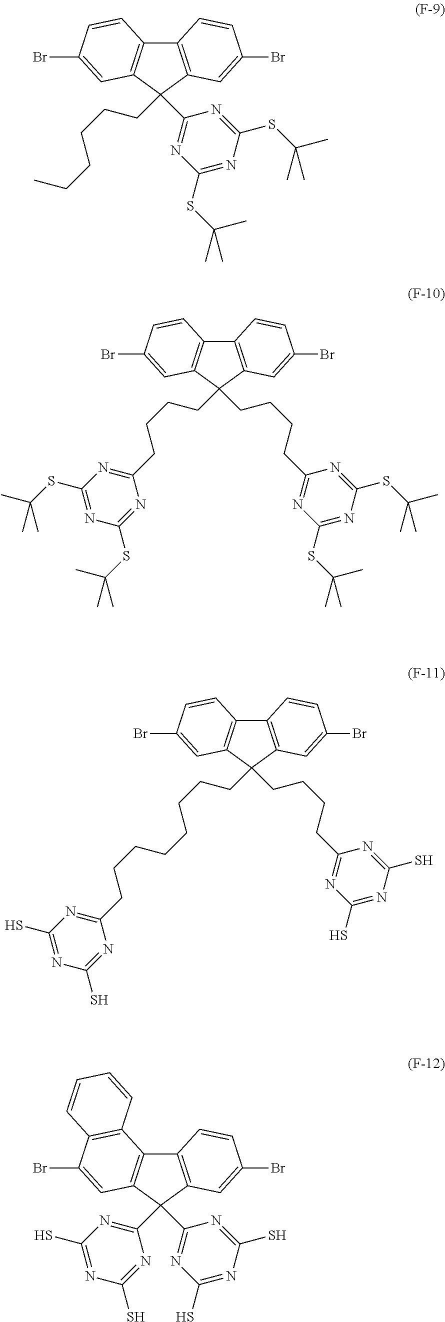

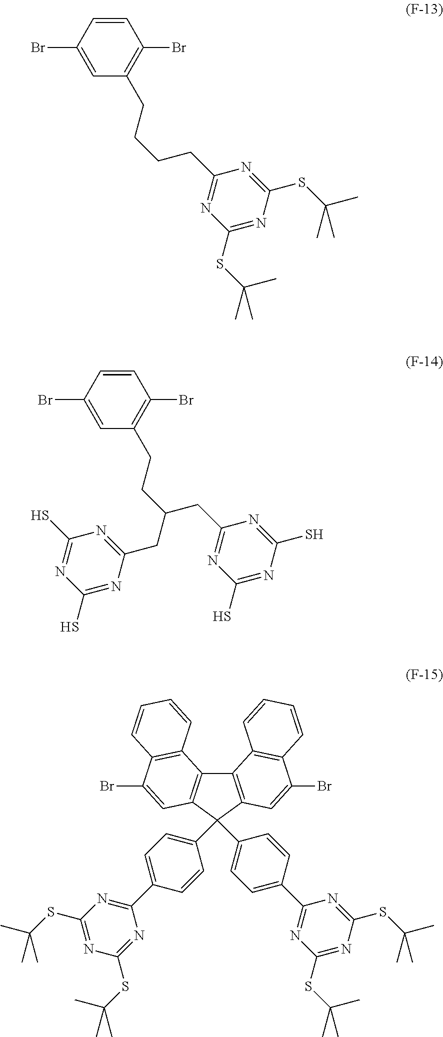

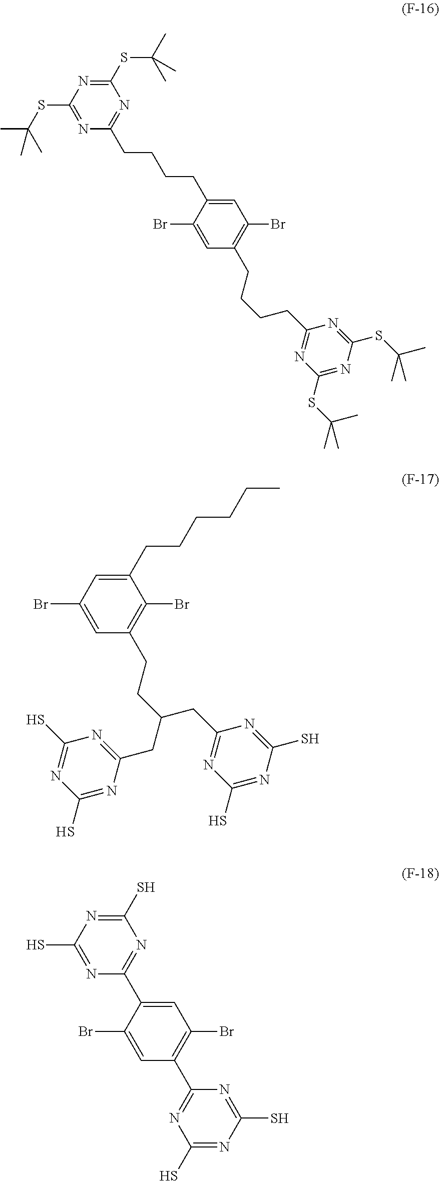 [SMILES: CC(C)(C)Sc1nc(CCCCc2cc(Br)c(CCCCc3nc(SC(C)(C)C)nc(SC(C)(C)C)n3)cc2Br)nc(SC(C)(C)C)n1 ; CCCCCCc1cc(Br)cc(CCC(Cc2nc(S)nc(S)n2)Cc2nc(S)nc(S)n2)c1Br ; Sc1nc(S)nc(-c2cc(Br)c(-c3nc(S)nc(S)n3)cc2Br)n1]

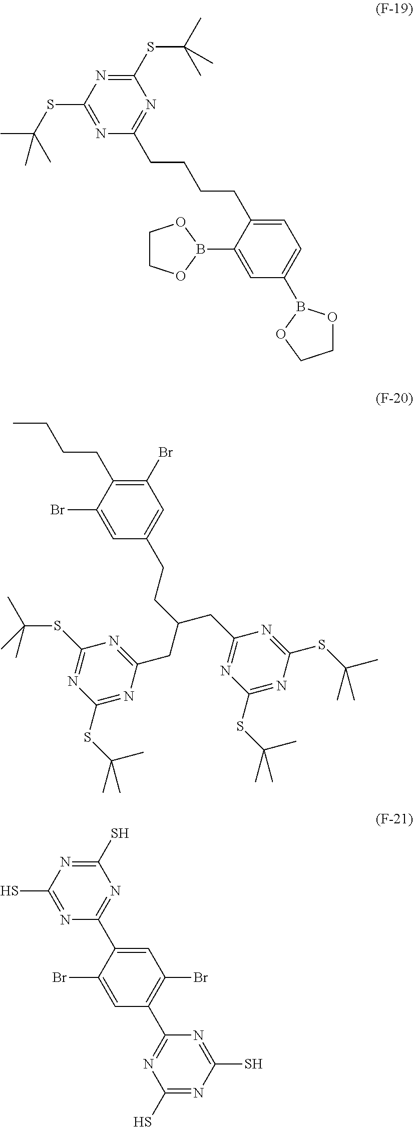

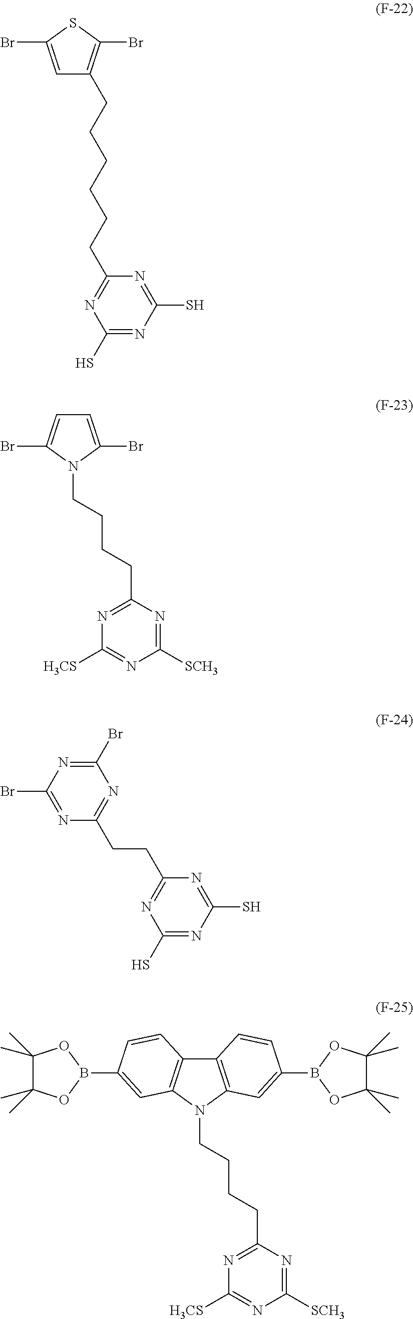

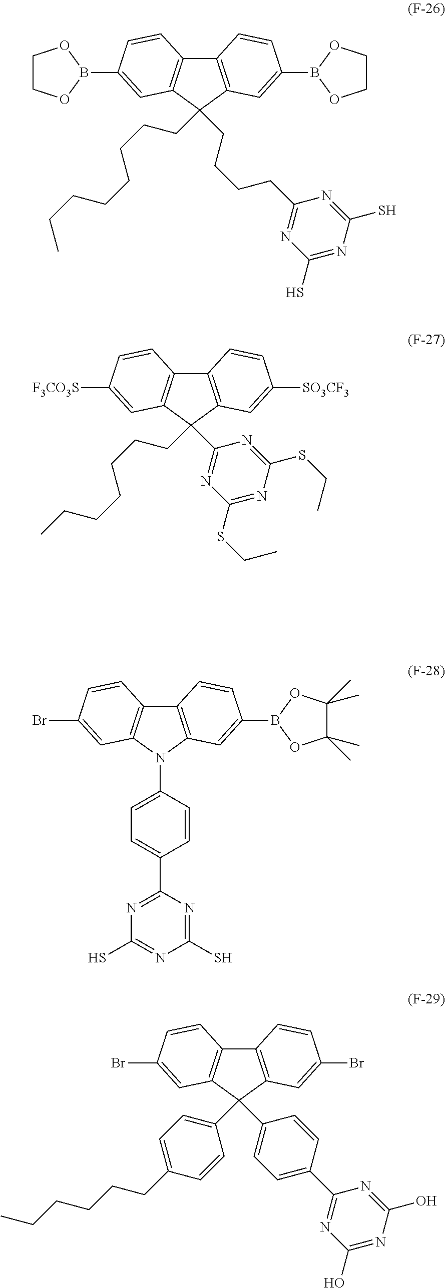

### <Production Method of Compound>

The compound of the present invention may be synthesized by any method, and one example of the synthesis method will be illustrated below (scheme A).

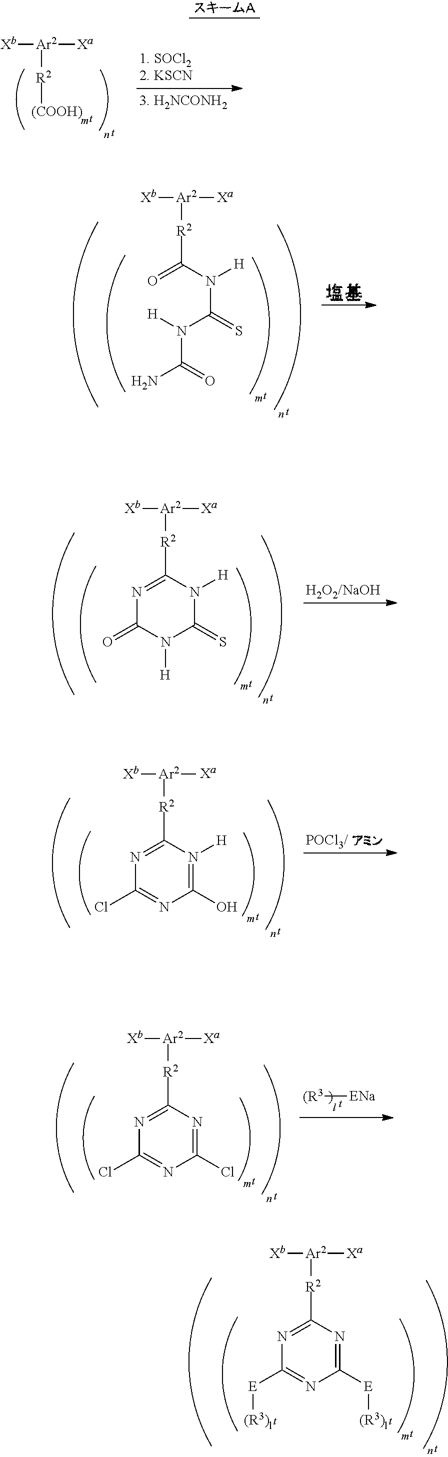

This scheme A can be applied when X^{a} and X^{b} represent a halogen atom. Here, when X^{a} and X^{b} represent —SO₃Q, —B(OQ¹)₂, —Si(Q²)₃ or Sn(Q³)₃ in the targeted compound of the scheme A, X^{a} and X^{b} may be advantageously converted into the correspondent group at a suitable stage in the scheme A. As the conversion method, a compound in which X^{a} and X^{b} represent a halogen atom (at any stage in the scheme A) is reacted with an organolithium such as n-butyllithium and the like, and thereafter,

(1) the compound is reacted with (Q¹O)₂B—B(OQ¹)₂, to obtain a compound in which at least one of X^{a} and X^{b} is converted into —B(OQ¹)₂,

(2) the compound is reacted with (Q²)₃SiCl, to obtain a compound in which at least one of X^{a} and X^{b} is converted into —Si(Q²)₃,

(3) the compound is reacted with (Q³)₃SnCl or (Q³)₃Sn—Sn(Q³)₃, to obtain a compound in which at least one of X^{a} and X^{b} is converted into —Sn(Q³)₃.

If each reaction in these conversion methods is carried out in the presence of a palladium catalyst, the reaction speed increases. When X^{a} and X^{b} represent an iodine atom or a bromine atom, the above-described organolithium reaction can be omitted.

Another example of the synthesis method of the compound of the present invention is a method of reacting a compound represented by the following formula (II-t) and a compound represented by the following formula (II-u).

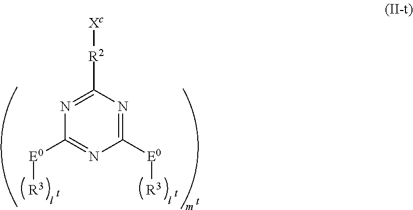

(wherein R², R³, E, l^{t} and m^{t} have the same meaning as described above. X^{c} represents a halogen atom, a nitro group, —SO₃Q, —B(OQ¹)₂, —B(OQ^{0 1})₃.M^{a}, —Si(Q²)₃ or —Sn(Q³)₃. When there exist a plurality of groups in parentheses appended with m^{t}, these may be mutually the same or different.)

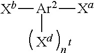

(wherein Ar², X^{a}, X^{b} and n^{t} have the same meaning as described above. X^{d} represents a halogen atom, a nitro group, —SO₃Q, —B(OQ¹)₂, —B(OQ^{0 1})₃.M^{a}, —Si(Q²)₃ or —Sn(Q³)₃.).

When X^{c} in the above-described formula (II-t) and X^{d} in the above-described formula (II-u) represent a halogen atom, the Kumada-Tamao coupling can be used. That is, a compound represented by the above-described formula (II-a) can be obtained by previously reacting magnesium or an alkylmagnesium chloride or the like with a compound represented by the above-described formula (II-t) or a compound represented by the above-described formula (II-u), thereby converting X^{c} or X^{d} into —MgX^{c}, —MgX^{d} or MgCl, then, reacting with the other compound in the presence of a nickel catalyst (e.g., NiCl₂(dppe)₂) or a palladium catalyst (e.g., Pd(PPh₃)₄).

When X^{c} in the above-described formula (II-t) and X^{d} in the above-described formula (II-u) represent a halogen atom, the Yamamoto coupling can also be used. That is, a compound represented by the above-described formula (II-a) can be obtained by coupling of reacting a compound represented by the above-described formula (II-t) and a compound represented by the above-described formula (II-u) in the presence of a nickel catalyst (e.g., bis(1,5-cyclooctadiene)nickel(0)).

When one of X′ in the above-described formula (II-t) and X^{d} in the above-described formula (II-u) is a halogen atom and the other is —B(OQ¹)₂ or B(OQ_{0 1})₃.M^{a}, the Suzuki-Miyaura coupling can be used. That is, a compound represented by the above-described formula (II-a) can be obtained by coupling of reacting a compound represented by the above-described formula (II-t) and a compound represented by the above-described formula (II-u) in the presence of a base and a palladium catalyst (e.g., Pd(PPh₃)₄).

Additionally, a compound represented by the above-described formula (II-a) can be obtained by subjecting a compound represented by the above-described formula (II-t) and a compound represented by the above-described formula (II-u) to the Ullmann reaction, the Glaser reaction, the Mizoroki-Heck reaction, the Negishi coupling, the Stille coupling, the Sonogashira coupling, the Buchwald-Hartwig reaction and the like.

### <Polymer Compound>

The polymer compound of the present invention is a polymer compound having a constituent unit represented by the above-described formula (P-a) and having a molecular weight of 5×10² to 1×10⁷. The polymer compound of the present invention is preferably a conjugated polymer compound since charges transfer easily in the molecule.

The molecular weight of the polymer compound of the present invention is preferably 1×10³ to 2×10⁶, more preferably 2×10³ to 1×10⁶, further preferably 2×10³ to 5×10⁵ since electric conductivity and coatability are excellent. The molecular weight of the resultant polymer compound is not uniform in some cases, and it is difficult to measure correct molecular weight in some cases. In this case, the number-average molecular weight or the weight-average molecular weight is obtained from distribution of the molecular weight reduced by a standard polymer compound such as polystyrene or the like using GPC (gel permeation chromatography), and used as the molecular weight of the conjugated compound.

Ar², R², E, R³, m^{t}, n^{t} and l^{t} in the above-described formula (P-a) are the same as those explained and exemplified as Ar², R², E, R³, m^{t}, n^{t} and l^{t} in the above-described formula (II-a). Examples of the constituent unit represented by the above-described formula (P-a) include groups obtained by removing two atoms or groups corresponding to X^{a} and X^{b} of compounds represented by the above-described formulae (F-1) to (F-28).

The constituent unit represented by the above-described formula (P-a) is preferably a constituent unit represented by the following formula (P-b), more preferably a constituent unit represented by the following formula (P-c) since electric conductivity, HOMO energy level, LUMO energy level and easiness of synthesis are improved.

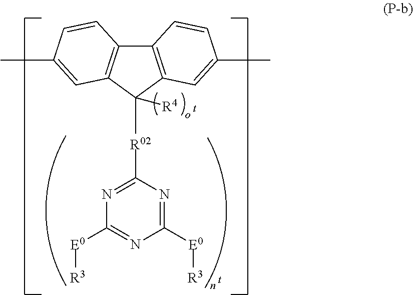

(wherein R^{0 2} represents a divalent hydrocarbon group, E⁰ represents a sulfur atom or an oxygen atom, R⁴ represents a monovalent hydrocarbon group or a hydrogen atom, n^{t} is 1 or 2, and o^{t}=2−n^{t}. R³ has the same meaning as described above. Each of a plurality of R³s and E⁰s may be mutually the same or different.

When there are a plurality of R^{0 2}s, these may be mutually the same or different. When there exist a plurality of groups in parentheses appended with n^{t}, these may be mutually the same or different.).

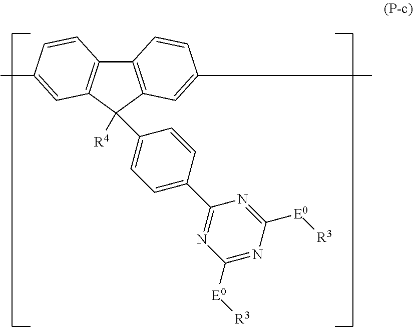

(wherein R³, R⁴ and E⁰ have the same meaning as described above.).

R^{0 2}, E⁰, R³, R⁴, o^{t} and n^{t} in the above-described formula (P-b) are the same as those explained and exemplified as R^{0 2}, E⁰, R³, R⁴, o^{t} and n^{t} in the above-described formula (II-b).

R^{0 2} in the above-described formula (P-b) is preferably a phenylene group.

E⁰, R³ and R⁴ in the above-described formula (P-c) are the same as those explained and exemplified as E⁰, R³ and R⁴ in the above-described formula (II-c).

The polymer compound of the present invention may be a homopolymer composed only of the constituent unit explained above, or a copolymer containing other constituent units. The other constituent units include constituent units represented by the formula —Ar²— such as a constituent unit composed of a dioctylfluorenediyl group, a constituent unit composed of a bithiophenediyl group and the like (this constituent unit is different from the constituent unit represented by the above-described formula (P-a)), etc.

When the polymer compound of the present invention is a copolymer, the number of the constituent unit represented by the above-described formula (P-a) contained in one molecule of the polymer compound is preferably 1 to 2000, more preferably 1 to 1000, further preferably 1 to 200, particularly preferably 1 to 50, especially preferably 1 to 20 since adsorption onto metal nano particles or metal compound nano particles becomes strong and solubility into a solvent is improved.

### <Production Method of Polymer Compound>

The polymer compound of the present invention may be synthesized by any method, and one example of the synthesis method is explained below by a method using the compound of the present invention.

For the polymer compound of the present invention, for example, the compound of the present invention may be singly used and reacted, alternatively the compound of the present invention and a compound represented by the following formula (V-a) and/or a compound represented by the following formula (V-b) may be used together and reacted. Each of these compounds may be used singly or in combination.

X^{b}—Ar²—X^{a} (V-a)

(wherein X′, X^{b} and Ar² have the same meaning as described above.).

H—Ar²—X^{a} (V-b)

(wherein X^{a} and Ar² have the same meaning as described above.).

When the compound of the present invention is singly used and reacted, the resulting polymer compound is a polymer compound composed only of a constituent unit represented by the above-described formula (P-a). In contrast, when the compound of the present invention and a compound represented by the formula (V-a) and/or a compound represented by the formula (V-b) are used together and reacted, the resulting polymer compound is a polymer compound containing a constituent unit represented by the following formula (P-V-a) and/or a group represented by the following formula (P-V-b).

—Ar²— (P-V-a)

(wherein Ar² has the same meaning as described above.).

—H—Ar²— (P-V-b)

(wherein Ar² has the same meaning as described above.).

The resulting polymer compound tends to have a linear structure containing groups represented by Ar² connected in chain form, and a group represented by the above-described formula (P-V-b) is contained as its end. When the compound represented by the above-described formula (V-a) and/or the compound of the present invention (compound represented by the above-described formula (II-a)) further has a group represented by —X^{a}, the resulting polymer compound has a structure of dendric form or network form.

When X^{a} and X^{b} in the compound of the present invention represent a halogen atom, the Kumada-Tamao coupling can be used as a reaction for obtaining the polymer compound of the present invention. That is, the polymer compound of the present invention can be obtained by previously reacting magnesium or an alkylmagnesium chloride or the like with the compound of the present invention, thereby converting X^{a} or X^{b} into —MgX^{a}, —MgX^{b} or MgCl, then, reacting in the presence of a nickel catalyst (e.g., NiCl₂(dppe)₂) or a palladium catalyst (e.g., Pd(PPh₃)₄). When a compound represented by the above-described formula (V-a) and/or a compound represented by the above-described formula (V-b) are allowed to coexist in the system, also X^{a} and X^{b} in these compounds preferably represent a halogen atom.

When X^{a} and X^{b} in the compound of the present invention represent a halogen atom, the Yamamoto coupling can also be used. That is, the polymer compound of the present invention can be obtained by coupling of reacting the compound of the present invention in the presence of a nickel catalyst (e.g., bis(1,5-cyclooctadiene)nickel(0)). When a compound represented by the above-described formula (V-a) and/or a compound represented by the above-described formula (V-b) are allowed to coexist in the system, also X^{a} and X^{b} in these compounds preferably represent a halogen atom.

When one of X^{a} and X^{b} in the compound of the present invention represents a halogen atom, and

(1) the other represents —B(OQ¹)₂ or B(OQ_{0 1})₃.M^{a},

(2) when X^{a} and X^{b} in the compound of the present invention represent a halogen atom, and X^{a} and X^{b} in the coexisting compound represented by the above-described formula (V-a) and X^{a} in the coexisting compound represented by the above-described formula (V-b) represent —B(OQ¹)₂ or B(OQ^{0 1})₃.M^{a}, or

(3) when X^{a} and X^{b} in the compound of the present invention represent —B(OQ¹)₂ or B(OQ^{0 1})³.M^{a}, and X^{a} and X^{b} in the coexisting compound represented by the above-described formula (V-a) and/or both X^{a} and X^{b} in the coexisting compound represented by the above-described formula (V-b) represent a halogen atom,

then, the Suzumi-Miyaura coupling can be used. That is, the polymer compound of the present invention can be obtained by coupling of reacting the compound of the present invention in the presence of a base and a palladium catalyst (e.g., Pd(PPh₃)₄).

Additionally, the polymer compound of the present invention can be obtained by subjecting the compound of the present invention to the Ullmann reaction, the Glaser reaction, the Mizoroki-Heck reaction, the Negishi coupling, the Stille coupling, the Sonogashira coupling, the Buchwald-Hartwig reaction and the like.

It is preferable for the polymer compound of the present invention that R³ in the above-described formula (P-a) is a hydrogen atom, since the compound can be adsorbed more efficiently and more strongly onto metal nano particles or metal compound nano particles. When the above-described synthesis method is applied, however, the coupling reaction and the like do not progress easily in some cases if a compound in which R³ is a hydrogen atom is used as the compound of the present invention. In this case, it is preferable that a polymer compound is synthesized using the compound of the present invention in which R³ is not a hydrogen atom, then, R³ in the above-described formula (P-a) is converted into a hydrogen atom using aluminum chloride, a carboxylic acid such as formic acid and the like, a sulfonic acid such as trifluoromethanesulfonic acid and the like; etc.

### <Metal Composite>

The metal composite of the present invention is a metal composite obtained by bringing the polymer compound of the present invention into contact with a metal in the form of film or plate or a metal compound in the form of film or plate or with metal nano particles having an aspect ratio of less than 1.5 or metal compound nano particles having an aspect ratio of less than 1.5.

The thickness of the above-described metal in the form of film or plate or the above-described metal composite in the form of film or plate is usually 0.01 nm to 10 cm, preferably 0.01 nm to 0.5 cm, more preferably 0.01 nm to 200 μm, further preferably 0.01 nm to 20 μm.

The above-described metal or the metal constituting the above-described metal compound includes aluminum, scandium, titanium, vanadium, chromium, manganese, iron, cobalt, nickel, copper, zinc, gallium, germanium, yttrium, zirconium, niobium, molybdenum, ruthenium, rhodium, palladium, silver, cadmium, indium, tin, antimony, lanthanum, cerium, europium, hafnium, tantalum, tungsten, rhenium, osmium, iridium, platinum, gold, mercury, thallium, lead and bismuth, preferably aluminum, titanium, vanadium, chromium, manganese, iron, cobalt, nickel, copper, zinc, gallium, germanium, yttrium, zirconium, molybdenum, ruthenium, rhodium, palladium, silver, indium, tin, antimony, lanthanum, cerium, tantalum, tungsten, iridium, platinum, gold, lead and bismuth, further preferably aluminum, titanium, vanadium, chromium, manganese, iron, cobalt, nickel, copper, zinc, gallium, rhodium, palladium, silver, indium, tin, tungsten, iridium, platinum, gold and lead, particularly preferably aluminum, chromium, manganese, iron, cobalt, nickel, copper, rhodium, palladium, silver, indium, tin, iridium, platinum and gold.

The metal compound includes alloys, metal oxides, composite oxides, metal nitrides, metal sulfides and metal salts, preferably alloys, metal oxides, composite oxides and metal sulfides.

The metal compound includes indium tin oxide (ITO), indium zinc oxide (IZO), molybdenum oxide, aluminum oxide, titanium oxide, zinc oxide, copper oxide, copper (II) oxide, magnesium oxide, yttrium oxide, tungsten(VI) oxide, silicon oxide, tin(IV) oxide, nickeltungsten, cerium oxide, manganese oxide, tin sulfide, cobalt oxide, holmium oxide, tricobalt tetroxide, triiron tetroxide, cobalt aluminate (CoAl₂O₄), spinel (Al₂O₃/MgO) and the like, preferably indium tin oxide, indium zinc oxide, molybdenum oxide, aluminum oxide, titanium oxide, zinc oxide, copper oxide, copper (II) oxide, magnesium oxide, yttrium oxide, tungsten(VI) oxide, silicon oxide and tin(IV) oxide, more preferably indium tin oxide (ITO), indium zinc oxide (IZO), molybdenum oxide, aluminum oxide, titanium oxide and zinc oxide.

The above-described metal in the form of film or plate or the above-described metal oxide in the form of film or plate can be fabricated by molding such as metallic casting, metallic rolling, facing, polishing and the like, vacuum film formation such as vapor deposition, sputtering, ion plating and the like, plating treatments such as electroplating, anodization, nonelectrolytic plating, chemical plating and the like, coating such as application of a particle dispersion liquid and the like, and preferable are those fabricated by metallic rolling, vapor deposition, sputtering and ion plating.

The above-described metal nano particles and metal composite nano particles have a length of the longest axis of usually 10 μm or less, preferably 0.1 nm to 1 μm, more preferably 1 nm to 500 nm.

The above-described metal nano particles and metal compound nano particles have an aspect ratio (namely, denoting longest diameter/shortest diameter, and when the aspect ratio has distribution, the average value thereof) of less than 1.5, preferably 1.4 or less, more preferably 1.3 or less, further preferably 1.2 or less, particularly preferably 1.1 or less.

The metal nano particles may be composed of the above-described metal itself or may be composed of a compound obtained by adsorption of other substance onto the above-described metal. The above-described metal compound nano particles may be composed of the above-described metal compound itself or may be composed of a compound obtained by adsorption of other substance onto the above-described metal compound.

The substance which can be adsorbed on the above-described metal and the above-described metal compound includes alkanethiols having 1 to 50 carbon atoms (e.g., tetradecanethiol, dodecanethiol, decanethiol, octanethiol), inorganic porous bodies, polyacrylamide, polyvinylpyrrolidone, polyacrylic acid and the like. At least a part of these substances are substituted by the polymer compound of the present invention in the production step of a metal composite.

The above-described contact can be carried out by (1) a method in which the polymer compound of the present invention is pasted to a metal in the form of film or plate or a metal compound in the form of film or plate, (2) a method in which the polymer compound of the present invention is applied onto a metal in the form of film or plate or a metal compound in the form of film or plate, (3) a method in which the polymer compound of the present invention and metal nano particles or metal compound nano particles are stirred and kneaded. In this contact, it is preferable that a solvent is allowed to mediate. Further, in this contact, other components may coexist in the system, or an ultrasonic wave may be applied.

In the case of the above-described method (2), it is preferable that a solution containing the polymer compound of the present invention in an amount of 0.0001 to 50 wt % is prepared and the solution is applied onto a metal in the form of film or plate or a metal compound in the form of film or plate.

When a solvent is allowed to coexist in contact in the case of the above-described method (3), those which are capable of dissolving the polymer compound of the present invention and do not dissolve metal nano particles or metal compound nano particles are usually used as the solvent. This solvent includes methanol, ethanol, benzyl alcohol, acetone, methyl ethyl ketone, dimethylformamide, dimethyl sulfoxide, ethyl acetate, toluene, xylene, orthodichlorobenzene, chloroform, tetrahydrofuran, hexane, benzene, diethyl ether, acetonitrile, acetic acid, water, propanol, butanol and N-methylpyrrolidone, and from the standpoint of the solubility of a conjugated compound, preferably includes methanol, ethanol, benzyl alcohol, acetone, methyl ethyl ketone, dimethylformamide, dimethyl sulfoxide, ethyl acetate, toluene, xylene, chloroforth, tetrahydrofuran, benzene, acetonitrile, propanol, butanol and N-methylpyrrolidone. The solvents may be used singly or in combination.

In the case of the above-described method (3), the stirring and kneading temperature is preferably −70° C. to 200° C., more preferably −10° C. to 120° C., further preferably 0° C. to 100° C., particularly preferably 20° C. to 70° C.

In the case of the above-described method (3), the stirring and kneading time is preferably 0.01 second to 1000 minutes, more preferably 0.1 second to 900 minutes, further preferably 1 second to 500 minutes.

As a method of confirming that the polymer compound of the present invention is adsorbed onto a metal or a metal compound by the above-described contact, it is preferable to measure a change in the binding energy of a chemical bond (covalent bond, coordination bond, hydrogen bond), a change in the atom electron density, a change in the energy level of the electron orbital and the like, ascribable to the mutual action between the polymer compound and the metal or metal compound. The method for measuring these changes includes infrared spectroscopy, near-infrared spectroscopy, raman spectroscopy, UV-visual absorption spectroscopy, fluorescence spectroscopy, phosphorescence spectroscopy, photoelectron spectroscopy, differential scanning calorimetry (DSC), thermogravimetry (TG), nuclear magnetic resonance (NMR) and the like. These changes appear as an apparent color change in some cases. Formation of a metal composite can also be confirmed by a change in solubility in some cases.

A step of purifying the resultant composite (hereinafter, referred to as “purification step”) may be carried out after the above-described contact.

In the case of the above-described method (2), a solvent can be removed by heating and drying the resultant composite. When a surplus amount of the polymer compound is adsorbed onto the resultant composite, the surplus polymer compound can be removed by spraying a solvent which is capable of dissolving the polymer compound, or putting the resultant composite into a solvent.

In the case of the above-described method (3), the surplus polymer compound can be removed by subjecting the resultant composite to ultrasonic dispersion, centrifugal separation, decantation, redispersion, dialysis, filtration, washing, heating, drying and the like.

### <Electron Device>

In the metal composite of the present invention, charge transfer is easy between the polymer compound and a metal or a metal compound. Therefore, in an electron device containing the metal composite of the present invention, namely, in an electron device in which the polymer compound of the present invention and a metal or a metal compound are laminated in the form of layer, charge transfer is easy between layers, and the electric current flowing in the device increases. The electron device of the present invention includes light emitting devices such as organic EL devices and the like, transistors, photoelectric conversion devices such as solar batteries and the like, etc.

### EXAMPLES

The present invention will be illustrated using examples below, but the present invention is not limited to them.

### Example 1

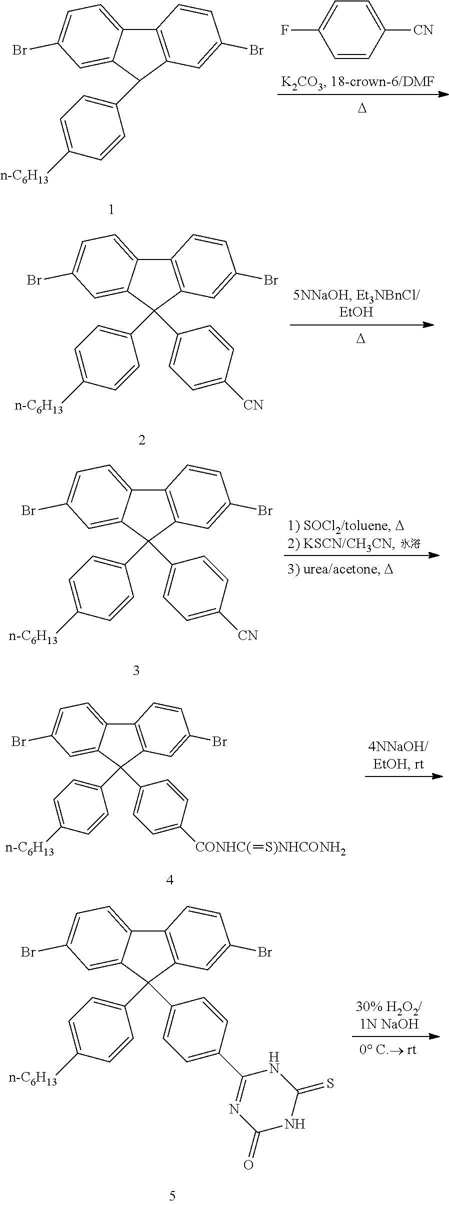

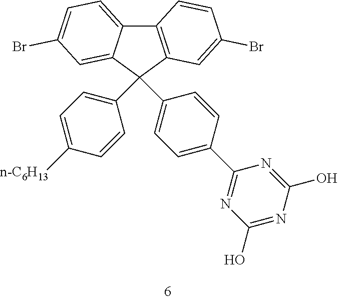

### [Synthesis of Compound 2]

Under a nitrogen atmosphere, a DMF suspension (164 ml) of 16.4 g (33.9 mmol) of a compound 1 (M1a described in JP-A No. 2009-149850, Example 1), 4.11 g (34.0 mmol) of 4-fluorobenzonitrile, 9.36 g (67.8 mmol) of potassium carbonate and 8.95 g (33.9 mmol) of 18-crown-6 was stirred with heating at 135 to 140° C. for 25 hours. After cooling, the mixture was diluted with water (492 ml), and 1N hydrochloric acid (20.3 ml) was added and the mixture was extracted with ethyl acetate and the liquid was separated. The aqueous layer was extracted with ethyl acetate, the combined organic layers were washed with saturated saline, then, the organic layer was dried over anhydrous magnesium sulfate. The residue obtained by concentration under reduced pressure was purified by silica gel chromatography (SiO₂, 600 g, hexane:ethyl acetate=30:1→25:1 (volume ratio)) to obtain 13.3 g (22.7 mmol) of a compound 2 as a crystal. The yield was 67.2%. The structure of this compound 2 was confirmed by NMR.

### [Synthesis of Compound 3]

Under a nitrogen atmosphere, a mixture of 13.9 g (23.8 mmol) of the compound 2, 1.81 g (7.96 mmol) of triethyl benzyl ammonium chloride, a 5N sodium hydroxide aqueous solution (192 ml) and ethanol (192 ml) was refluxed with heating for 16.5 hours. After cooling, the mixture was neutralized with concentrated hydrochloric acid, and ethanol was removed under reduced pressure. Concentrated hydrochloric acid was added to the mixture to render pH thereof to 2, and the mixture was extracted with t-butyl methyl ether and the liquid was separated. The aqueous layer was extracted with t-butyl methyl ether, the combined organic layers were washed with saturated saline, then, the organic layer was dried over anhydrous magnesium sulfate. Under reduced pressure, the mixture was concentrated to obtain 13.8 g (22.9 mmol) of a compound 3 as a crystal. The yield was 96.2%. The structure of this compound 3 was confirmed by NMR.

### [Synthesis of Compound 4]

Under a nitrogen atmosphere, to a mixture of 12.6 g (20.9 mmol) of the compound 3 and toluene (38 ml) was added 20.6 g (173 mmol) of thionyl chloride, and the mixture was heated at 80° C. for 1 hour. After cooling, the mixture was concentrated under reduced pressure, to the resultant residue was added toluene, and further concentrated under reduced pressure, and this series of operations were repeated three times, then, the mixture was further dried under reduced pressure to obtain 13.2 g (21.2 mmol) of an acid chloride. The apparent yield was 101%.

Under a nitrogen atmosphere, an acetonitrile solution (51 ml) of 2.27 g (23.4 mmol) of potassium isothiocyanate was dropped into a toluene solution (12 ml) of 12.1 g (19.5 mmol) of the above-described acid chloride over a period of 15 minutes while cooling in an ice bath. After completion of dropping, the mixture was stirred at the same temperature for 1 hour. The resultant mixture was filtrated, and the filtrate was concentrated under reduced pressure. To the resultant residue was added acetone (47 ml), and further, 1.40 g (23.3 mmol) of urea was added and the mixture was stirred with heating at the reflux temperature for 3 hours. After cooling, the mixture was concentrated under reduced pressure, and water was added to cause deposition of a crystal which was then filtrated. The crystal was washed with water and dried under reduced pressure, to obtain 11.1 g (15.8 mmol) of a compound 4 as a crystal. The yield was 81.0%. The structure of this compound 4 was confirmed by NMR.

### [Synthesis of Compound 5]

Under a nitrogen atmosphere, a mixture of 10.2 g (14.5 mmol) of the compound 4, ethanol (136 ml) and a 4N sodium hydroxide aqueous solution (272 ml) was stirred at room temperature overnight. 1M sulfuric acid was added to render pH thereof to 6 while cooling in an ice bath, and the deposited crystal was filtrated. The crystal was washed with water, and dried under reduced pressure, to obtain 10.3 g (15.0 mmol) of a compound 5 as a crystal. The apparent yield was 103%. The structure of this compound 5 was confirmed by NMR.

### [Synthesis of Compound 6]

Under a nitrogen atmosphere, into a mixture of 10.3 g (15.0 mmol) of the compound 5 and a 1N sodium hydroxide aqueous solution (121 ml) was dropped 30 wt % hydrogen peroxide water (30.3 ml) while cooling in an ice bath. The mixture was stirred at the same temperature for 15 minutes, then, stirred at room temperature for 30 minutes. Disappearance of raw materials was confirmed by TLC, then, 1M sulfuric acid was used to render pH thereof to 7 while cooling in an ice bath. The deposited crystal was filtrated, the crystal was washed with water, and a mixed solvent of hexane and ethyl acetate (hexane:ethyl acetate=2:1 (volume ratio)), then, dried under reduced pressure, to obtain 6.67 g (9.94 mmol) of a compound 6 as a crystal. The yield was 66.3%. The structure of this compound 6 was confirmed by NMR.

### Example 2

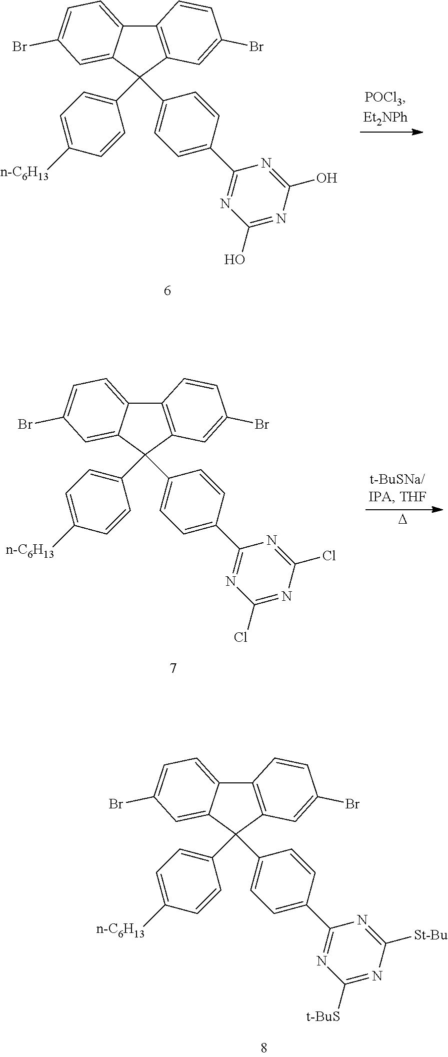

### [Synthesis of Compound 7]

Under a nitrogen atmosphere, a mixture of 6.61 g (9.85 mmol) of the compound 6, 19.8 ml (212 mmol) of phosphorus oxychloride and 1.51 g (10.1 mmol) of N,N-diethylaniline was stirred with heating at 105° C. for 3 hours. After cooling, excess phosphorus oxychloride was removed under reduced pressure, to the residue were added chloroform and water and the mixture was stirred at room temperature for 1 hour. The separated organic layer was washed with water, a sodium hydrogen carbonate aqueous solution (to render pH of the aqueous layer approximately to 7) and saturated saline in this order, and the organic layer was dried over anhydrous magnesium sulfate. The crystal obtained by concentration under reduced pressure was further dried under reduced pressure, to obtain 7.00 g (9.85 mmol) of a compound 7. The yield was 100%. The structure of this compound 7 was confirmed by NMR.

### [Synthesis of Compound 8]

Under a nitrogen atmosphere and an ice bath, to 0.91 g (20.9 mmol) of 55 wt % sodium hydride was added dehydrated isopropyl alcohol (210 ml), and the mixture was stirred at room temperature until sodium hydride disappeared and generation of hydrogen stopped. While cooling with an ice bath again, 2.54 ml (22.5 mmol) of t-butylmercaptane was dropped. After completion of dropping, a dehydrated THF solution (37 ml) of 7.00 g (9.89 mmol) of the compound 7 was dropped at room temperature. The resultant mixture was stirred with heating at the reflux temperature for 2 hours. After cooling, the resultant mixture was concentrated under reduced pressure, to the resultant residue was added hexane, the deposited crystal was removed by filtration, washed with hexane and acetonitrile, and dried under reduced pressure to obtain a compound 8 (4.40 g). The filtrate was concentrated, and the resultant residue was purified by silica gel chromatography (SiO₂, 30 g, hexane→hexane:toluene=8:1→6:1 (volume ratio)), and the compound 8 was combined to obtain 5.46 g (6.70 mmol) of a crystal (liquid chromatography purification: 97.2%). Recrystallization was performed using about 50 milliliters of a mixed solvent of hexane and chloroform (hexane:chloroform=10:1 (volume ratio)), and the crystal was washed with hexane twice, to obtain 3.0 g of a compound 8. The yield was 37.2%. The structure of this compound 8 was confirmed by NMR.

### Example 3

### Synthesis of Polymer Compound P-1

Into a 50 ml flask were charged 100 mg (0.12 mmol) of the compound 8, 560 mg (1.06 mmol) of a compound represented by the following formula:

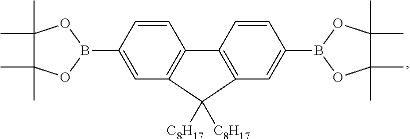

, 496 mg (0.86 mmol) of a compound represented by the following formula:

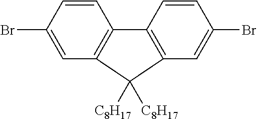

and 74 mg of a phase transfer catalyst (trade name: Aliquat336 (registered trademark)(manufactured by Aldrich)), and an atmosphere in the flask was purged with an argon gas. Next, a solution prepared by adding 227 mg (0.196 mmol) of tetrakis(triphenylphosphine)palladium to 20 mL of toluene was added and the mixture was stirred, and 10.0 mL of a 0.60 mol/L sodium carbonate aqueous solution was added and the mixture was stirred. Next, the mixture was stirred at 100° C. for 5 hours. Thereafter, the mixture was cooled down to room temperature, then, the organic layer and the aqueous layer of the reaction solution were separated, and the organic layer was dropped into 100 mL of methanol to cause deposition of a precipitate, and the precipitate was filtrated and dried, to obtain 717 mg of a polymer compound J as a solid.

According to the NMR results, the polymer compound J has two kinds of repeating units represented by the following formulae.

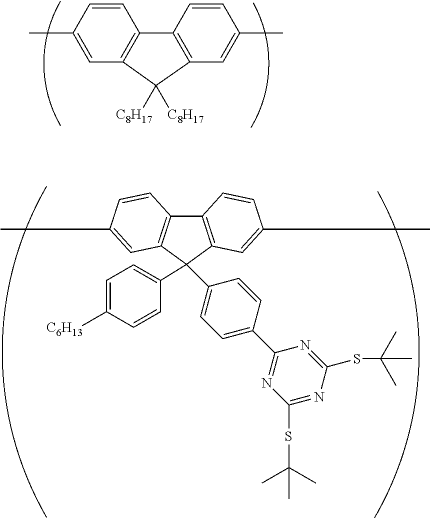

The amount of a constituent unit represented by the following formula:

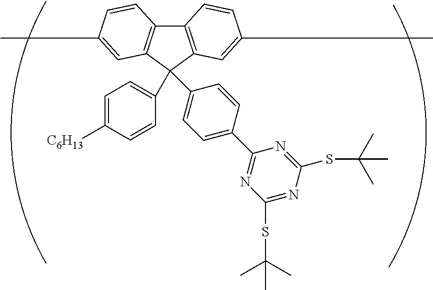

in the polymer compound J was 4.4 mol %. The polymer compound J had a polystyrene-equivalent number-average molecular weight of 6.7×10³ and a polystyrene-equivalent weight-average molecular weight of 1.3×10⁴.

Into a 50 ml flask were charged 485 mg of the polymer compound J and 31.5 ml of toluene, and the mixture was stirred at room temperature. Next, aluminum chloride was added, then, the mixture was stirred at 115° C. for 1 hour. After cooling, the organic layer in the reaction vessel was dropped into 100 ml of methanol, to cause deposition of a precipitate. The precipitate was filtrated and dried, to obtain 260 mg of a solid. Based on the NMR analysis results, it was confirmed that a signal derived from a t-butyl group of the polymer compound J disappeared completely. The solid is believed as a polymer compound P-1 (polymer) having two kinds of repeating units represented by the following formulae.

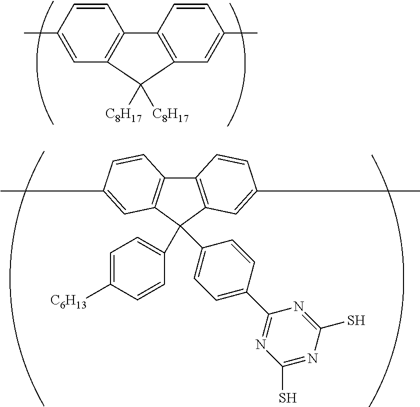

The polymer compound P-1 had a polystyrene-equivalent number-average molecular weight of 6.6×10³ and a polystyrene-equivalent weight-average molecular weight of 1.6×10⁴.

### Example 4

### Synthesis of Polymer Compound P-2

Into a 50 ml flask were charged 100 mg (0.12 mmol) of the compound 8, 158 mg (0.24 mmol) of a compound represented by the following formula:

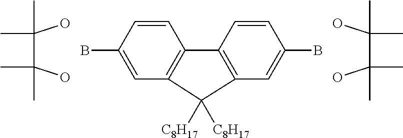

67.4 mg (0.12 mmol) of a compound represented by the following formula:

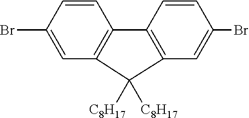

and 19.3 mg of a phase transfer catalyst (trade name: Aliquat336 (registered trademark)(manufactured by Aldrich)), and an atmosphere in the flask was purged with an argon gas. Into this was charged 8 mL of toluene, and the mixture was stirred at 30° C. for 5 minutes. Next, 14.9 mg (0.048 mmol) of tetrakis(triphenylphosphine)palladium was added and the mixture was stirred at 30° C. for 10 minutes, and 4.0 mL of a 2N sodium carbonate aqueous solution was added, then, the mixture was stirred at 30° C. for 5 minutes. Next, the mixture was stirred at 100° C. for 8 hours. Thereafter, the mixture was cooled down to room temperature, then, the organic layer and the aqueous layer of the reaction solution were separated, and the organic layer was dropped into 200 mL of methanol to cause deposition of a precipitate, and the precipitate was filtrated and dried, to obtain a yellow solid. This yellow solid was charged into a 300 ml flask, and 100 mL of toluene was added to cause dissolution thereof, and the solution was stirred at 30° C. for 5 minutes. Next, 10 g of activated carbon was charged and the mixture was stirred at 100° C. for 2 hours. Thereafter, the mixture was cooled down to room temperature, then, the organic layer was filtrated, and concentrated to 5 ml. The concentrated organic layer was dropped into 200 ml of methanol to cause deposition of a precipitate, and the precipitate was filtrated and dried, to obtain 100 mg of a polymer compound G.

According to the NMR results, the polymer compound G has two kinds of repeating units represented by the following formulae.

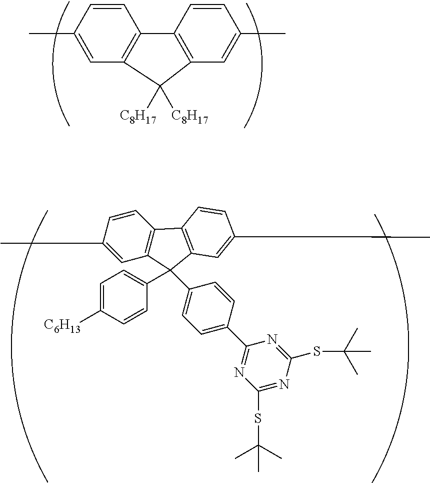

The amount of a constituent unit represented by the following formula:

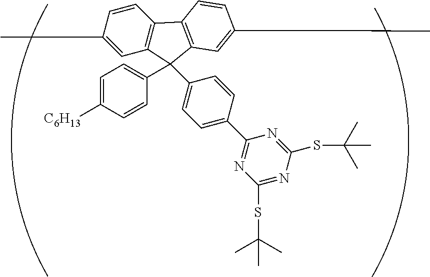

in the polymer compound G was 15 mol %. The polymer compound G had a polystyrene-equivalent number-average molecular weight of 7.9×10³ and a polystyrene-equivalent weight-average molecular weight of 1.9×10⁴.

Into a 50 ml flask were charged 80 mg of the polymer compound G and 20 ml of toluene, and the mixture was stirred at room temperature for 10 minutes. Next, aluminum chloride was added, then, the mixture was further stirred for 1 hour. The organic layer in the reaction vessel was dropped into 500 ml of methanol, to cause deposition of a precipitate. The precipitate was filtrated and dried, to obtain 40 mg of a solid. Base on the NMR analysis results, it was confirmed that a signal derived from a t-butyl group of the polymer compound G disappeared completely. The solid is believed as a polymer compound P-2 (polymer) having two kinds of repeating units represented by the following formulae.

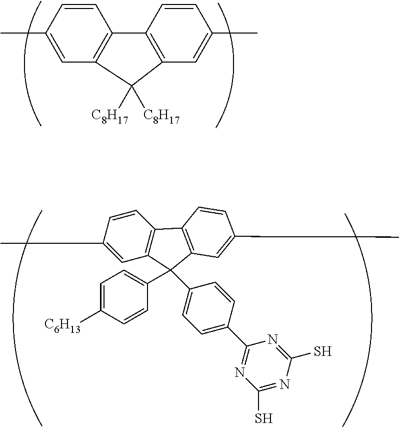

The polystyrene-equivalent number-average molecular weight and weight-average molecular weight of the polymer compound P-2 were the same as those of the polymer compound G.

### Example 5

### Synthesis of Polymer Compound P-3

Into a 50 ml flask were charged 100 mg (0.12 mmol) of the compound 8, 410 mg (0.98 mmol) of a compound represented by the following formula:

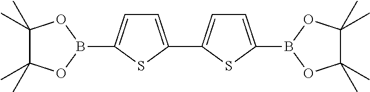

496 mg (0.86 mmol) of a compound represented by the following formula:

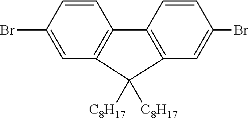

and 19.3 mg of a phase transfer catalyst (trade name: Aliquat336 (registered trademark) (manufactured by Aldrich)), and an atmosphere in the flask was purged with an argon gas. Into this was charged 20 mL of a toluene solution containing 227 mg (0.20 mmol) of tetrakis(triphenylphosphine)palladium dissolved therein and the mixture was stirred. Next, 10.0 mL of a 0.59 mol/L sodium carbonate aqueous solution was added, then, the mixture was stirred at 100° C. for 5.5 hours. Thereafter, the mixture was cooled down to room temperature, then, the organic layer and the aqueous layer of the reaction solution were separated, and the organic layer was dropped into 200 mL of methanol to cause deposition of a precipitate, and the precipitate was filtrated and dried, to obtain 606 mg of a polymer compound K.

According to the NMR results, the polymer compound K is represented by the following formula.

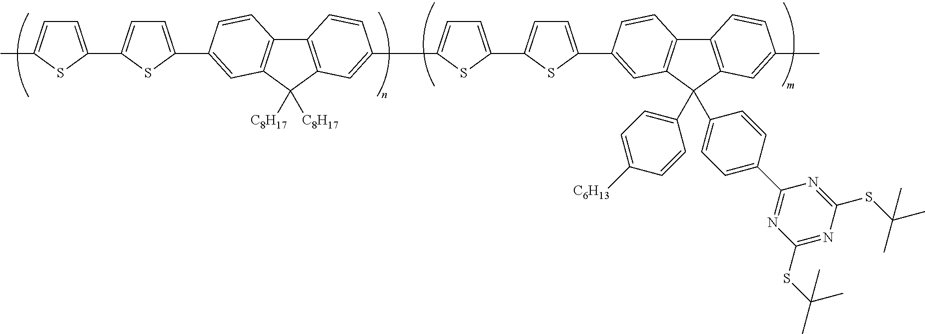

Compound K
   (wherein n and m are a number representing the number of repeating units. n:m is guessed to be 7:1 according to the charged ratio.).

The polymer compound K had a polystyrene-equivalent number-average molecular weight of 3.5×10³ and a polystyrene-equivalent weight-average molecular weight of 1.1×10⁴.

Into a 50 ml flask were charged 300 mg of the polymer compound K and 3.9 ml of toluene, the mixture was cooled with an ice bath, then, 0.6 mL of trifluoromethanesulfonic acid and 0.6 mL of trifluoroacetic acid were added, then, the mixture was heated at 80° C. and stirred for 7 hours. After cooling down to room temperature, the organic layer in the reaction vessel was dropped into 100 ml of methanol to cause deposition of a precipitate. The precipitate was filtrated and dried, dissolved in chloroform, and this solution was dropped into 100 ml of methanol to cause deposition of a precipitate. The precipitate was filtrated and dried, to obtain 250 mg of a solid. Based on the NMR analysis results, it was confirmed that a signal derived from a t-butyl group of the polymer compound G disappeared completely. The solid is believed as a polymer compound P-3 (polymer) represented by the following formula.

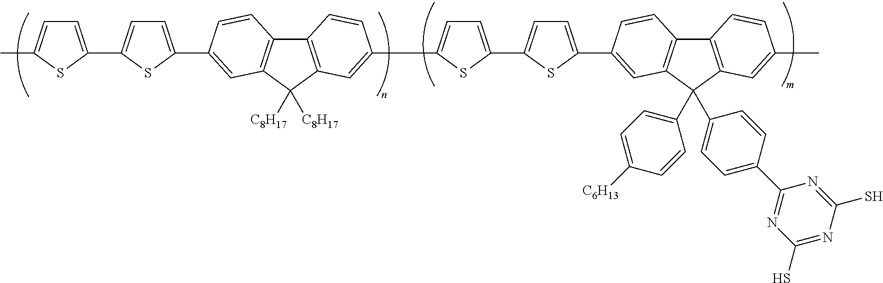

(wherein n and m are a number representing the number of repeating units. n:m is guessed to be 7:1 according to the charged ratio.).

The polymer compound P-3 had a polystyrene-equivalent number-average molecular weight of 4.3×10³ and a polystyrene-equivalent weight-average molecular weight of 1.5×10⁴.

### Example 6

The polymer compound P-1 (6.7 mg) was dissolved in 3 mL of toluene. From 3 mL of a hexane solution of silver nano particles of which surface had been modified with dodecanethiol (particle size (DLS): 5-15 nm, 0.25% (w/v) hexane solution, manufactured by Aldrich), hexane was distilled off by an evaporator, 3 mL of toluene was added, this was mixed with the toluene solution of the polymer compound P-1, and the mixture was allowed to stand still for 1.5 hours. This solution was stable, and a precipitate was not generated. This was spin-coated (previously filtrated through a pore filter, 500 rpm, 2 minutes), to fabricate a film having a thickness of 10 nm. This is a composite composed of the polymer compound P-1 and the silver nano particles.

### Example 7

Silver nano particles (nano powder, particle size: <100 nm, 99.5% trace metals basis, manufactured by Aldrich)(7 mg) were added to 1.5 mL of toluene, and placed together with the vessel into un ultrasonic wave washing machine and the silver particles were diffused by an ultrasonic wave. At this point, the liquid kept gray turbid condition for a while, and one hour after, the silver nano particle precipitated and the supernatant became clear. Again, the silver particles were diffused by an ultrasonic wave, then, 2 mg of the polymer compound P-1 was added and the mixture was stirred, then, the resultant dispersion was allowed to stand still, then, the dispersion remained turbid even after one hour and the silver particles were diffused. It was understood from this result that the silver nano particle were stabilized by adsorption of the polymer compound P-1 onto the surface of the silver particle.

### Example 8

The polymer compound P-3 (8.6 mg) was dissolved in 2.2 g of toluene to prepare a toluene solution of the polymer compound P-3. From a hexane solution of silver nano particles of which surface had been modified with dodecanethiol (particle size (DLS): 5-15 nm, 0.25% (w/v) hexane solution, manufactured by Aldrich), hexane was distilled off by an evaporator (here, the weight of the silver nano particles was 8.6 mg), and 2.2 g of toluene was added to prepare a toluene solution of the silver nano particles.

An aliquot (1.32 g) of the toluene solution of the silver nano particles and an aliquot (1.29 g) of the toluene solution of the polymer compound P-3 were mixed. The resultant mixed solution was transparent and uniform, and a precipitate was not generated. When this was dropped into methanol, a brown precipitate was generated and the supernatant was colorless and transparent.

In contrast, when the toluene solution of the silver nano particles was dropped into methanol, a precipitate was not generated and the brown transparent liquid was uniform. Further, when the toluene solution of the polymer compound P-3 was dropped into methanol, a yellow precipitate was generated and the supernatant was colorless and transparent.

In consideration of these results, it is believed that by mixing of the toluene solution of the silver nano particles with the toluene solution of the polymer compound P-3, dodecanethiol on the surface of the silver nano particles is substituted by the polymer compound P-3, and in methanol, the silver nano particles precipitate together with the polymer compound P-3, and the silver nano particles are not present in the liquid. This precipitate is a composite composed of the polymer compound P-3 and the silver nano particles. This precipitate was separated from the methanol solution using a centrifugal separator, dried and the ¹H-NMR spectrum thereof (in deuterated chloroform, TMS standard) was measured, to find no signal ascribable to dodecanethiol. In contrast, when toluene was distilled off from the above-described toluene solution of the silver nano particles by an evaporator and the ¹H-NMR spectrum thereof (in deuterated chloroform, TMS standard) was measured, signals ascribable to dodecanethiol could be confirmed (for example, 2.66 ppm is characteristic).

In consideration of these results, it was understood that by mixing of the toluene solution of the silver nano particles with the toluene solution of the polymer compound P-3, most of dodecanethiol on the surface of the silver nano particles is substituted by the polymer compound P-3, a composite composed of the polymer compound P-3 and the silver nano particles is generated, and the polymer compound P-1 is adsorbed strongly onto the silver nano particles.

1. A compound represented by the following formula (II-a): 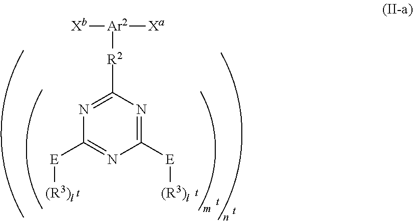
(wherein Ar² represents an aromatic group optionally having a substituent, R² represents a direct bond or an organic group optionally having only an oxygen atom as a hetero atom, E represents a hetero atom, R³ represents a monovalent hydrocarbon group or a hydrogen atom, m^{t} and n^{t} represent each independently an integer of 1 or more, l^{t} represents an integer of 1 to 3. Each of a plurality of R³s, Es and l^{t}s may be mutually the same or different. When there exist a plurality of m^{t} s, these may be mutually the same or different. X^{a} and X^{b} represent each independently a halogen atom, a nitro group, —SO₃Q (here, Q represents an unsubstituted or substituted monovalent hydrocarbon group.), —B(OQ¹)₂ (here, Q¹ represents a hydrogen atom or a monovalent hydrocarbon group, alternatively two Q¹s are linked together to form a ring. Two Q¹s may be mutually the same or different.), —B(OQ^{0 1})₃.M^{a} (wherein, Q^{0 1} represents a hydrogen atom or a monovalent hydrocarbon group, alternatively two to three Q⁰¹s are linked together to form a ring. Three Q⁰¹s may be mutually the same or different. M^{a} represents a metal cation or an ammonium cation optionally having a substituent.), —Si(Q²)₃ (here, Q² represents a monovalent hydrocarbon group.) or —Sn(Q³)₃ (here, Q³ represents a monovalent hydrocarbon group.). When there exist a plurality of groups in parentheses appended with m^{t} and n^{t}, these may be mutually the same or different.).

2. The compound according to claim 1, wherein the compound is represented by the following formula (II-b): 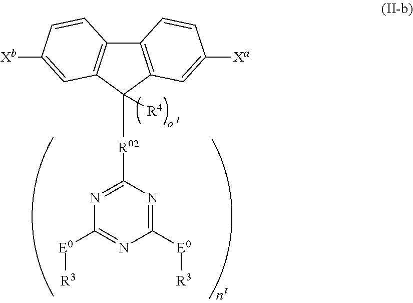
(wherein R^{0 2} represents a divalent hydrocarbon group, and E⁰ represents a sulfur atom or an oxygen atom. R⁴ represents a monovalent hydrocarbon group or a hydrogen atom, n^{t} is 1 or 2, and o^{t}=2−n^{t}. Each of a plurality of R³s and E⁰s may be mutually the same or different. When there exist a plurality of R^{0 2} s, these may be mutually the same or different. R³, X^{a} and X^{b} have the same meaning as described above. When there exist a plurality of groups in parentheses appended with n^{t}, these may be mutually the same or different.).

3. The compound according to claim 2, wherein R^{0 2} represents a phenylene group.

4. The compound according to claim 2, wherein X^{a} and X^{b} represent each independently a chlorine atom, a bromine atom or an iodine atom.

5. The compound according to claim 4, wherein the compound is represented by the following formula (II-c): 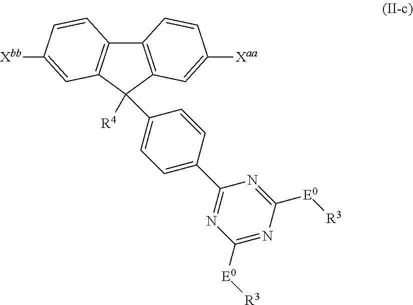
(wherein R³, R⁴ and E⁰ have the same meaning as described above. X^{a a} and X^{b b} represent each independently a chlorine atom, a bromine atom or an iodine atom.).

6. A polymer compound having a constituent unit represented by the following formula (P-a) and having a molecular weight of 5×10² to 1×10⁷: 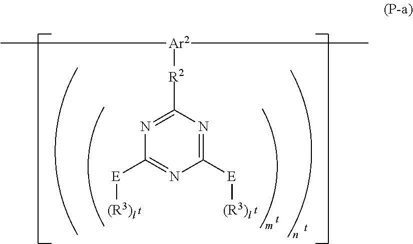
(wherein Ar² represents an aromatic group optionally having a substituent, R² represents a direct bond or an organic group optionally having only an oxygen atom as a hetero atom, E represents a hetero atom, R³ represents a monovalent hydrocarbon group or a hydrogen atom, m^{t} and n^{t} represent each independently an integer of 1 or more, and l^{t} represents an integer of 1 to 3. Each of a plurality of R³s, Es and l^{t}s may be mutually the same or different. When there exist a plurality of m^{t} s, these may be mutually the same or different. When there exist a plurality of groups in parentheses appended with m^{t} and n^{t}, these may be mutually the same or different.).

7. The polymer compound according to claim 6, wherein the constituent unit represented by said formula (P-a) is a constituent unit represented by the following formula (P-b): 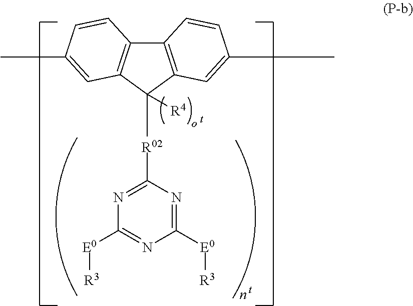
(wherein R^{0 2} represents a divalent hydrocarbon group, E⁰ represents a sulfur atom or an oxygen atom, R⁴ represents a monovalent hydrocarbon group or a hydrogen atom, n^{t} is 1 or 2, and o^{t}=2−n^{t}. R³ has the same meaning as described above. Each of a plurality of R³s and E⁰s may be mutually the same or different. When there exist a plurality of R^{0 2} s, these may be mutually the same or different. When there exist a plurality of groups in parentheses appended with n^{t}, these may be mutually the same or different.).

8. The polymer compound according to claim 7, wherein R^{0 2} represents a phenylene group.

9. The polymer compound according to claim 8, wherein the constituent unit represented by said formula (P-b) is a constituent unit represented by the following formula (P-c): 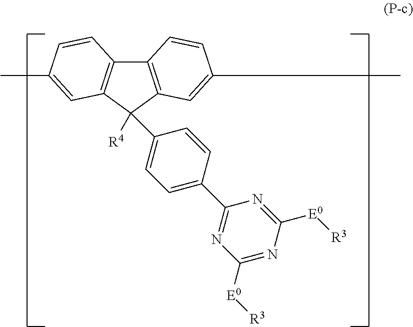
(wherein R³, R⁴ and E⁰ have the same meaning as described above.).

10. A metal composite obtained by bringing the polymer compound according to claim 6 into contact with a metal in the form of film or plate or with a metal compound in the form of film or plate.

11. A metal composite obtained by bringing the polymer compound according to claim 6 into contact with metal nano particles having an aspect ratio of less than 1.5 or with metal compound nano particles having an aspect ratio of less than 1.5.

12. An electron device comprising the metal composite according to claim 10.

13. An electron device comprising the metal composite according to claim 11.

14. An electron device comprising a metal composite obtained by bringing the polymer compound according to claim 6 into contact with a metal in the form of film or plate or with a metal compound in the form of film or plate, and into contact with metal nano particles having an aspect ratio of less than 1.5 or with metal compound nano particles having an aspect ratio of less than 1.5.
